(19) United States
(12) Patent Application Publication
(10) Pub. No.: US 2003/0087309 A1
(43) Pub. Date: May 8, 2003

(54) Desktop drug screening system

(76) Inventor: Shiping Chen, Fremont, CA (US)

Correspondence Address: Shantanu Basu, Morrison & Foerster LLP, 755 Page Mill Road, Palo Alto, CA, 94304-1018, US

(21) Appl. No.: 10/229,571

(22) Filed: Aug. 27, 2002

Related U.S. Application Data
(63) Continuation-in-part of application No. 10/080,274, filed on Feb. 19, 2002.
(60) Provisional application No. 60/315,285, filed on Aug. 27, 2001.
(60) Provisional application No. 60/327,686, filed on Oct. 4, 2001.
(60) Provisional application No. 60/357,275, filed on Feb. 15, 2002.

(51) Int. Cl. B01J 19/00; G01N 33/48

(52) U.S. Cl. 435/7.1; 422/68.1; 436/518

(57) ABSTRACT

Systems and methods involved in extreme high throughput screening of compounds which have an affinity for a biological target are disclosed. The system is based on a capillary bundle with two distinguishable ends wherein capillaries on one end are connected to compounds stored in discrete reservoirs and capillaries on the other end are bound and processed to form a two dimensional microarray. A capillary bundle having reaction wells for hybridization and compound reaction in one end of the capillaries is disclosed. Also disclosed are various methods of identifying a target compound in a liquid using this capillary bundle as well as methods of fabricating the bundle. A novel surface tension guided reaction chamber is also provided. Methods and chemistry for fabrication and use of a surface tension guided reaction chamber in binding and hybridization assays are also disclosed. Methods and systems for precise metering of fluids within the capillaries and at the reaction chambers, including the surface tension guided “virtual” reaction well are provided.

### CROSS-REFERENCE TO RELATED APPLICATIONS

This application claims benefit of priority to U.S. Provisional Patent Application Serial No. 60/315,285, entitled “Desktop Drug Screening System,” filed Aug. 27, 2001; U.S. patent application Ser. No. 60/327,686, entitled “Single Use XHTS Chip”, filed Oct. 4, 2001; U.S. patent application Ser. No. 60/357,275, entitled “Reagent Metering,” filed Feb. 15, 2002, and U.S. patent application Ser. No. 10/080,274, entitled “Method and Apparatus Based on Bundled Capillaries for High Throughput Screening,” filed Feb. 19, 2002. All of the above applications are incorporated by reference herein in their entireties as if fully set forth below for all purposes.

### TECHNICAL FIELD OF THE INVENTION

The invention relates generally to the field of biochemical analysis. More specifically, the invention relates to biochemical analysis in which it is desirable to gauge the interaction of targets from of one or multiple solutions to probes. The invention provides methods, devices and compositions for high throughput screening (HTS), proteomics, and polymerase chain reaction (PCR) amplification.

### BACKGROUND OF THE INVENTION

High throughput screening (HTS) is a key step in drug discovery process. The process of drug discovery is critically dependent upon the ability of screening efforts to identify lead compounds with future therapeutic potential. The screening efforts are often described as one of the bottlenecks in the process of drug discovery. One strategy for identifying pharmaceutical lead compounds is to develop an assay that provides appropriate conditions for monitoring the activity of a therapeutic target for a particular disease. This assay is then used to screen large numbers of potential modulators of the therapeutic target in the assay. For example, libraries of chemical compounds can be screened in assays to identify their activity in relation to therapeutic targets and cells.

In “High Throughput Screening” (HTS), the reagents are enzymes and substrates while the entities are a library of chemical compounds. Biochemical and biological assays are designed to test for activity of chemical entities in a broad range of systems ranging including protein-protein interactions, enzyme catalysis, small molecule-protein binding and other cellular functions. In HTS one uses these kinds of assays to simultaneously test a large number of chemical entities in order to discover biological or biochemical activities of the chemical entities.

Existing HTS systems use standard microtiter plates as the basic liquid handling medium. The compound libraries are typically stored in dry powder form. Every certain period, say one year, solutions are made from the powders and stored in individual wells of standard microtiter plates with 96 or less wells. These plates are “mother plates”. Before screening, the compounds are transferred into plates with denser (384 or 1536) wells, i.e. “daughter plates”, to conserve reagents. Because a typical compound library comprises more than one million chemical compounds, it takes thousands of microtiter plates to complete the screening process. A large number of robotic systems are required to move and store the plates and to transfer and handle liquid samples. As a result, existing HTS systems occupy warehouse-sized space and are very expensive to acquire, operate and maintain. Because of the cost of operating the robotic systems and the logistic difficulty in distribution and tracking of these daughter plates, current HTS operation are conducted at a central location and only very large companies can afford to run such central facilities.

Current HTS technologies are based on microtitre plates (comprising 96, 384, or 1536 wells per plate) with most widely established techniques utilizing 96-well microtitre plates. In this format, 96 independent tests are performed simultaneously on a single 8 cm×12 cm plastic plate that contains 96 reaction wells. These wells typically require assay volumes that range from 50 to 500 μl. In addition to the plates, many instruments, materials, pipettes, robotics, plate washers and plate readers are commercially available to fit the 96-well format to a wide range of homogeneous and heterogeneous assays.

To date, efforts to improve HTS have generally focused on miniaturization. By reducing the well size the number of wells on each plate is increased in order to provide more parallel testing. Furthermore, by decreasing assay volumes, the cost of reagents is also reduced. Moreover, because more parallel tests can be run with smaller assay volumes, the simultaneous testing of more compounds to find drug candidates is speeded up. Miniaturization has marginally improved the 96-well technology by providing a 384-well format. (Comley et al., J. Biomol. Screening, 2(3):171-78 (1997)).

Assay development, on the other hand, is a highly distributed process. From small academic labs to units within a large organization, each researcher can develop an assay and would prefer to perform the screening in person so that he or she has the opportunity to fine-tune the assay and to better interpret and understand the results. The existing technology is not able to fulfill this requirement.

### SUMMARY OF THE INVENTION

The invention comprises a desktop drug screening system including equipment such as a desktop HTS station, a capillary loading station, a capillary array compound library, and combinations and subcombinations of these three systems.

In one embodiment, the invention provides a method for high throughput screening (HTS) of a compound library of one or more probes for a property of interacting with a target, the method comprising: providing the compound library in a capillary array comprising a plurality of channels assembled in a substrate, wherein each capillary channel is capable of holding an amount of a probe and further wherein the first ends of a plurality of channels form a first face of the capillary array; providing a reaction well adjacent one end of the capillary such that a probe in the capillary is capable of interacting with a target molecule in the reaction well; providing at least one target molecule in the reaction well; and detecting an interaction of a probe with the target molecule.

The reaction well may comprise a separate assay array assembled in a substrate, wherein the probe in a capillary array is capable of being in fluid communication with a channel in the assay array. In one embodiment, the assay array has an identical pitch and pattern of capillaries as the capillary array. In another embodiment, a first face of the assay array is coupled to the capillary array and a second face of the assay array is pneumatically coupled to a pressure chamber.

In one embodiment, the reaction well comprises a micro reaction well fabricated at a first end of each channel of the capillary array, wherein the probe in a capillary array is capable of being in fluid communication with the micro reaction well. In one embodiment, the reaction well comprises a virtual reaction well fabricated at a first end of each channel of the capillary array, wherein the reaction well is formed on the first face of the capillary array, the reaction well being defined by a hydrophilic region at the first end of the channel and a hydrophobic region surrounding the hydrophilic region.

In one embodiment, each capillary channel is capable of holding a metered amount of the probe. In another embodiment, the method comprises pumping a probe solution to the reaction well by applying a suitable pressure differential between the pressure chamber and the first face of the array. In another embodiment, the method comprises pumping a probe solution to the reaction well by inserting a liquid immiscible with the probe into the pressure chamber; and moving the probe solution between the channel and the reaction well by displacing a volume of the inert fluid in the pressure chamber.

The invention also provides a method for high throughput screening (HTS) of one or more probes for an enzymatic activity, the method comprising: (a) providing a capillary array comprising a plurality of channels assembled in a substrate, wherein each capillary channel is capable of holding an amount of a probe and further wherein the first ends of a plurality of channels form a first face of the capillary array; (b) providing a virtual reaction well adjacent one end of the capillary, wherein the reaction well is formed on the first face of the capillary array and further wherein the reaction well is defined by a hydrophilic region at the first end of the channel and a hydrophobic region surrounding the hydrophilic region; (c) applying a target solution to the first face of the capillary array in a flooding manner such that droplets of the target solution are retained in the reaction wells after excess solution is allowed to run off, (d) applying a negative pressure to a pressure chamber to draw a metered amount of substrate into the channel, wherein a second distal face of the array is coupled to the pressure chamber; (e) removing excess substrate fluid from the reaction well; (f) applying a metered amount of an enzyme to the reaction by a method comprising steps (c) through (e) wherein the solution contains the enzyme; (g) applying a positive pressure in the pressure chamber to push a metered amount of enzyme, target and compound into the micro-reaction well; and (h) detecting the enzymatic activity of a probe in a channel.

In one embodiment, the method comprises removing excess target solution from the reaction well by a method selected from the group consisting of capillary force, squeegeeing, wiping, absorption, gravity, centrifugation, air pressure, air knife blowing and vacuum force.

The invention provides a desktop high throughput screening (HTS) system for detecting a property of one or more probe compounds to interact with a target, the system comprising: (a) a compound library of probes in a capillary array comprising a plurality of channels assembled in a substrate, wherein each capillary channel is capable of holding an amount of a probe and further wherein the first ends of a plurality of channels form a first face of the capillary array; and a reaction well adjacent one end of the capillary such that a probe in the capillary is capable of interacting with a target molecule in the reaction well; and (b) a desktop HTS station comprising: a pressure chamber capable of connecting to the capillary array; a chamber for reacting metered amounts of probes and at least one target; and a detector for detecting an interaction of a probe with the target molecule. The system may further comprise: (c) a compound loading station comprising a plurality of probe compounds stored individually in a plurality of reservoirs, such that each reservoir is fluidically coupled to a channel in the capillary array. In one embodiment, the reaction well comprises a separate assay array assembled in a substrate, wherein the probe in a capillary array is capable of being in fluid communication with a channel in the assay array.

A novel surface tension guided reaction chamber is also provided. Methods and chemistry for fabrication and use of a surface tension guided reaction chamber in binding and hybridization assays are also disclosed. Methods and systems for precise metering of fluids within the capillaries and at the reaction chambers, including the surface tension guided “virtual” reaction well is provided.

Methods for performing high throughput screens using optical fiber lined capillaries of the invention are also provided. Chemical compounds could either be in solution in the capillary or immobilized on the walls of the capillary.

Interaction of the target and chemical compounds can be detected by fluorescence emission (intrinsic or extrinsic probes), fluorescence polarization, luminescence, absorption, surface plasmon resonance (SPR) or other signals of the target system. The detection system can be a CCD based fluorescence imaging system or a scanning based fluorescence system. In the second approach, absorption of samples can also be measured by placing a light source and a detector on different sides of the through hole plate.

### BRIEF DESCRIPTION OF THE DRAWINGS

FIG. 1 illustrates a configuration of the capillary array that is used to hold the compound library.

FIGS. 2a-2c illustrate liquid holding patterns in through holes.

FIGS. 3a-3b illustrate internal structures of through holes.

FIG. 4 illustrates a “virtual” micro-reaction well.

FIG. 5 illustrates a scheme for constructing capillary arrays with separate pieces of uniform through hole arrays.

FIG. 6 illustrates the basic configuration of capillary array substrate for the portable compound library.

FIG. 7 illustrates capillary array compound library in different formats. FIG. 7A illustrates a “branch” format. FIG. 7B illustrates a “bundle” format. FIG. 7C illustrates a “chip” format.

FIG. 8 illustrates internal structure of a through hole in a capillary array compound library.

FIG. 9 illustrates a number of structures of the compound storage chamber.

FIG. 10 illustrates a number of internal structures of mixing/reaction chamber.

FIG. 11 illustrates volume metering by surface tension patch.

FIG. 12 illustrates volume metering by a flow regulator with a side air tunnel linking the air above the mixing chamber to the narrow path.

FIG. 13 illustrates chamber volume metering by internal through hole structures with fluid barriers within the chamber. FIG. 13a illustrates that the barrier may be a short narrow opening. FIG. 13b illustrates that the barrier may be a short hydrophobic zone. FIG. 13c illustrates that the barrier may be an interface from a smaller to a larger chamber.

FIG. 14 illustrates process of metering multiple different reagents using multiple interconnected chambers.

FIG. 15 illustrates a through hole structure which comprises multiple chambers linked to a chamber in parallel.

FIG. 16 illustrates a method of excess fluid removal by vacuum.

FIG. 17 illustrates a method of excess fluid removal by a second capillary array.

FIG. 18 illustrates a method of excess fluid removal by wiping.

FIG. 19 illustrates a method for reducing cross-contamination between adjacent holes during excess fluid removal.

FIG. 20 illustrates a reaction chamber design using reflection wall of reaction chamber to enhance optical signal of an assay.

FIG. 21 illustrates another reaction chamber design using light guiding capillary to facilitate optical detection.

FIG. 22 illustrates a schematic diagram of a compound loading station.

FIGS. 23a-23b illustrate methods for loading compounds into capillary arrays. FIG. 23a depicts a single through hole aligned with cavities in capillary bundle of the loading station; and FIG. 23b depicts a capillary array with high-density small holes in comparison to the size and pitch of cavities in the delivery head of the loading station.

FIG. 24 is a perspective view of a capillary bundle in accordance with the present invention.

FIG. 25A illustrates one of the possible configurations of the compound loading station. A pressure chamber containing a compound library in microtiter plates is coupled to capillary bundles. When the pressure chamber is pressurized, compounds in the microtiter plates are delivered to the output ends of the capillary bundle where the assay will be conducted or loaded to another portable capillary array, which will be sent to users who will conduct HTS assay on a desktop screen station, as illustrated in FIG. 25B.

FIG. 26 illustrates another parallel fluid delivery method utilizing gravity as the driving force.

FIG. 27 illustrates one embodiment to fabricate the delivery head. FIG. 27a illustrates that the capillary tubes are first inserted into through holes of the guiding plate. FIG. 27b illustrates that a bonding material, such as epoxy or ceramic is used to solidify capillary tubes and the guide plate together. FIG. 27c illustrates that the solidified bundle is cut at a position very close to the guide plate. FIG. 27d illustrates that the end facet is polished and etched to form isolated “islands” of the tubes.

FIG. 28 illustrates a number of embodiments of fluidic features on the assay surface to prevent deposited solutions from cross contamination. FIG. 28a illustrates hydrophilic patches on the assay surface. FIG. 28b illustrates geometric structures, such as islands. FIG. 28c illustrates geometric structures, such as wells.

FIG. 29 illustrates schematics of a desktop extreme high throughput screening station.

FIGS. 30a-30h illustrate steps of a screening procedure according to the invention.

FIGS. 31a-3b illustrate methods of using a single-use screening chip.

FIG. 32 illustrates a delivery method using an intermediary through hole array.

FIG. 33 illustrates the operational steps for carrying out an enzymatic assay using a capillary array compound library designed for multiple uses. The through hole structure of the array comprises a micro-reaction well linked to a large compound reservoir through a long and narrow path.

FIG. 34 illustrates the operational steps for carrying out an enzymatic assay using a single use capillary array compound library. The through hole structure of the array comprises a “virtual well” on the assay surface.

FIG. 35 illustrates the operational steps for carrying out an enzymatic assay using a single use capillary array compound library. The through hole structure of the array comprises three interconnected chambers.

FIG. 36 illustrates a through hole metering plate.

FIG. 37 illustrates a capillary array cartridge having multiple chips within.

FIG. 38 illustrates an embodiment of an assay involving protein arrays or cell arrays.

### DETAILED DESCRIPTION OF THE INVENTION

This invention comprises various new systems and equipment, such as a desktop HTS station, a capillary loading station, a capillary array compound library, and combinations and subcombinations of these three systems. This invention thus includes methods and apparatus for performing HTS operation in a desktop system. It also includes the method and apparatus for the fabrication of such a system. This invention dramatically reduces complexity, cost and at the same time, significantly enhances the throughput in comparison with existing HTS systems.

The HTS system in one aspect of the invention utilizes: (i) a capillary array compound library; (b) a compound loading station; and (c) a desktop HTS station.

These three aspects are described in detail in the following sections. To use the HTS system described in the invention, the compound library is originally stored in mother plates at a central location. Compounds are first loaded into a miniature capillary array using the compound loading station. The volumes of compound solutions in each capillary array can vary from tens of microliters to less than a nanoliter depending on the size and configuration of the capillary array and are sufficient for a single or a fixed number of screening operations. The compact, capillary array based compound library is distributed to end users, who use the desktop HTS station to conduct high throughput screening operations.

(a) Capillary Array Compound Library

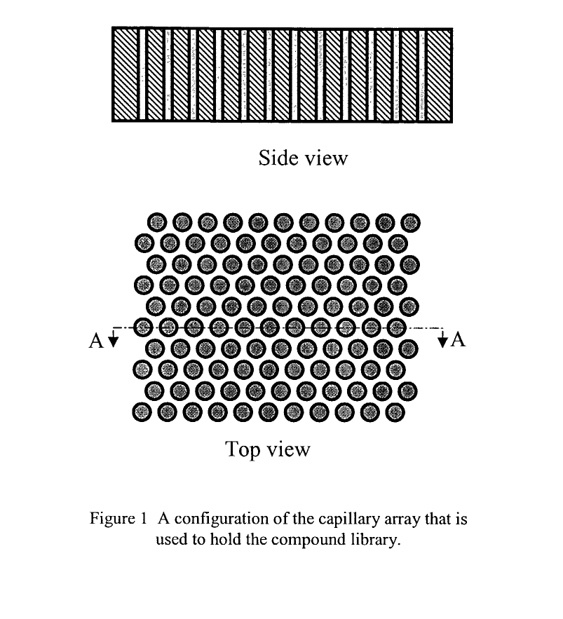

In this invention, the compound libraries used by HTS operators are stored in a special capillary array. Such an array comprises essentially a large number of through holes or channels grouped together in an integrated structure, as illustrated in FIG. 1. The structure can be rigid or flexible. The cross-sectional shapes of the holes are preferably circular but can also be any other shapes. The holes in an array can be the same or different in sizes. The spatial distribution of the holes in the array can be highly regular or completely random. The diameters of the holes can range from several nanometers to several tens millimeters. The length of the holes or channels can be from several micrometers to several meters. The pitch of the through holes in the array (distance from center to center of adjacent holes) can range from tens of nanometers to tens of millimeters.

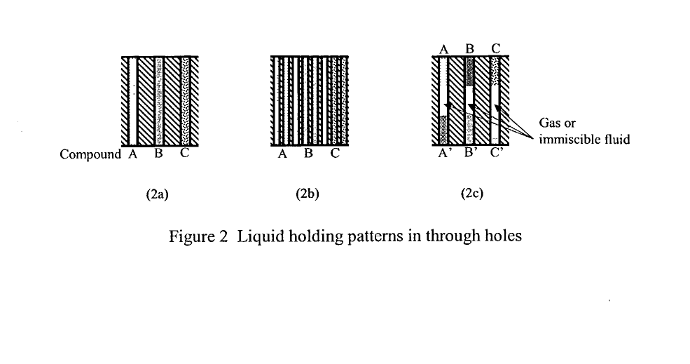

In a specific embodiment, the top and/or bottom surfaces of the array where the through holes exit are made hydrophobic and the inner surfaces of holes are made hydrophilic. Chemical compounds in liquid form are stored in the through holes of the array and held within by the capillary force. A compound can be individually held in a single hole as shown in FIG. 2a, or a single compound may be distributed in a number of different holes as shown in FIG. 2b. Alternatively, a single hole may store different compound and/or chemical solutions at different sections and these solutions are isolated from each other by a short section of gas or a liquid that is not dissolvable with the compound solution, as shown in FIG. 2c.

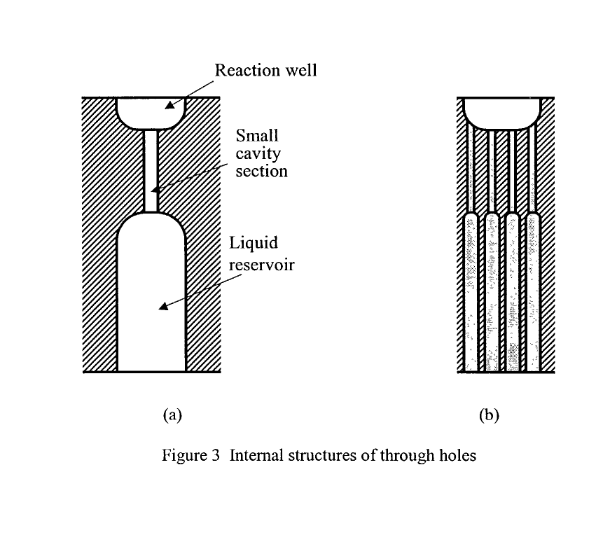

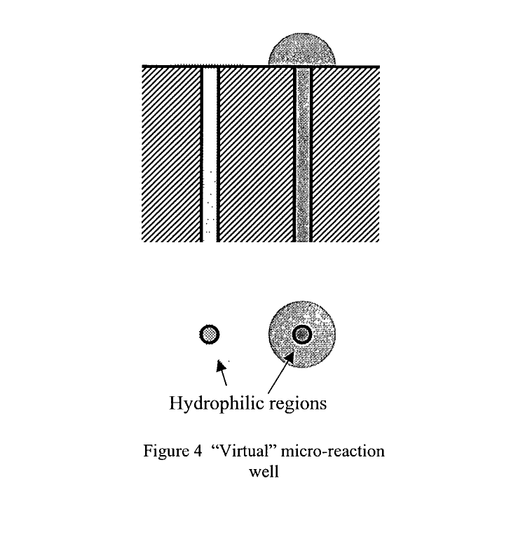

The liquid volume of each compound can be controlled by the diameter and/or length of the through hole or channel or the number of such holes that holds the compound as illustrated in FIGS. 2b and 3(b). The liquid volume may also be controlled by adjusting the length of a “slug” of fluid in the channel where a channel is not completely filled with a single fluid, as illustrated in FIG. 2c.

Alternatively, a section of the through hole can be enlarged to increase the compound volume without increasing the length of the through hole, as illustrated by the liquid reservoir in FIG. 3a. For example, a uniform through hole of 20 μm in diameter and 3 mm in length can hold 1 nl liquid. On the other hand, a 20 mm-long 20 μm diameter through hole having 16 mm of its length enlarged to a 90 μm diameter can hold as much as 12 μl compound solution. A 60 mm diameter capillary array with 100 μm through hole pitch can hold a compound library comprising up to 360,000 different solutions.

During transportation and distribution of the capillary array library, the two openings of each through hole may be sealed by a cap at each end of the array to prevent evaporation of the solutions.

The capillary array described above can be fabricated by several different methods. One method is to make each capillary individually using a preform extrusion process widely known in the art for working capillaries. Then a large number of such capillaries of the same or different channel diameter are assembled together by e.g. gluing or fusing them to form an array. The second method is to fabricate many through holes on a single piece of solid substrate material. The fabrication methods may include mechanical or laser drilling, or chemical/electrochemical etching by methods known in the art. The third method involves making a preform with many cavities in a much larger dimension, then extruding the preform to reduce the cavities to suitable hole or channel sizes. Methods as disclosed in U.S. Ser. No. 09/791,994, entitled Microarray Fabrication Technologies (Genestamp) filed on Feb. 22, 2001, U.S. Ser. No. 09/791,998, entitled Microarray Fabrication Techniques and Apparatus, filed Feb. 22, 2001, and U.S. Ser. No. 09/791,410, entitled Method and Apparatus Based on Bundled Capillaries for High Throughput Screening filed on Feb. 16, 2001, may be used. The fourth method uses a precision molding process to produce the array using known techniques. The material of the array can be silica, glass, ceramic or other metal oxide. Alternatively, the array can be made of plastic, metal, polymer or other suitable materials.

In one particular embodiment of this invention, the capillary array described here is only a compound storage medium. HTS assays are conducted in a separate “assay chip”, which has its own array of through holes. The assay chip may be made by forming a capillary bundle that has a total length equal to the desired length of the finished capillary array plus the desired length or thickness of the assay chip (plus any material loss due to cutting and/or polishing surfaces of the chip and array), and cutting the bundle to form both the assay chip and the capillary array.

In another embodiment, HTS assays are conducted directly on one end facet (termed “assay end”) of the capillary array. In this case, special “micro-reaction wells” are fabricated on the facet at the tip of each through holes. FIG. 3 illustrates two particular configurations of the micro-reaction wells, of which the diameter is substantially larger than that of the through holes. The compound held in the through hole may be mixed with the target reagents, such as the enzyme under investigation and the substrate, in the micro-reaction well. The mixture is incubated in and results read from the well. Such micro-reaction wells can be fabricated by etching and the etching pattern can be generated by lithographic techniques well known in the semiconductor industry. For example, a well of 100 μm in diameter and 50 μm deep can hold 0.4 nl fluid. By changing the diameter and depth of the wells, assay with different fluid volumes ranging from 0.5 μl to 0.1 nl can be conducted.

An alternative configuration of the micro-reaction well is a so-called “virtual well” as illustrated in FIG. 4, where the facet surface at the assay end is patterned as regions with different surface tensions. Hydrophilic regions are made around the holes and hydrophobic regions in the rest of the area. Each hydrophilic region around the through hole can hold a separate droplet of fluid, as shown in FIG. 4. Assuming a 90° contact angle, the volume of fluid that can be held within the hydrophilic region is 2πr3/3 where r is the radius of the semispherical droplet. A 100 μm diameter hydrophilic region in such a configuration can hold 0.25 nl solution. Such spatially patterned surface tension regions can be generated using lithographic methods. An alternative method to generate hydrophilic and hydrophobic regions at the assay end is to print a patterned layer of Teflon. The region covered by Teflon will be hydrophobic.

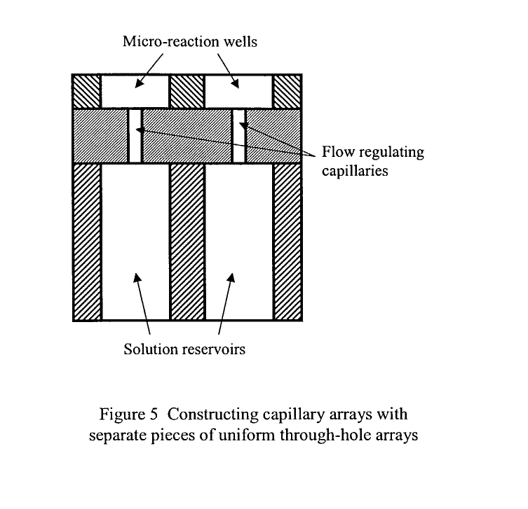

The micro-reaction wells, fluid reservoirs and the thin capillary linking the two can be fabricated out of a single piece of continuous material. Alternatively, they can be made out of separate pieces of materials, with each part having uniform through holes. Then the through holes in these three parts are aligned and assembled together, as illustrated in FIG. 5.

The configuration and method of fabrication of “real” micro-reaction well illustrated in FIG. 3 have been disclosed in GenoSpectra's patent application U.S. Ser. No. 09/791,411, entitled Liquid Arrays, filed on Feb. 22, 2001. The “virtual micro-reaction well” configuration illustrated above can also be used in protein array applications as discussed in the “Liquid Array” patent application.

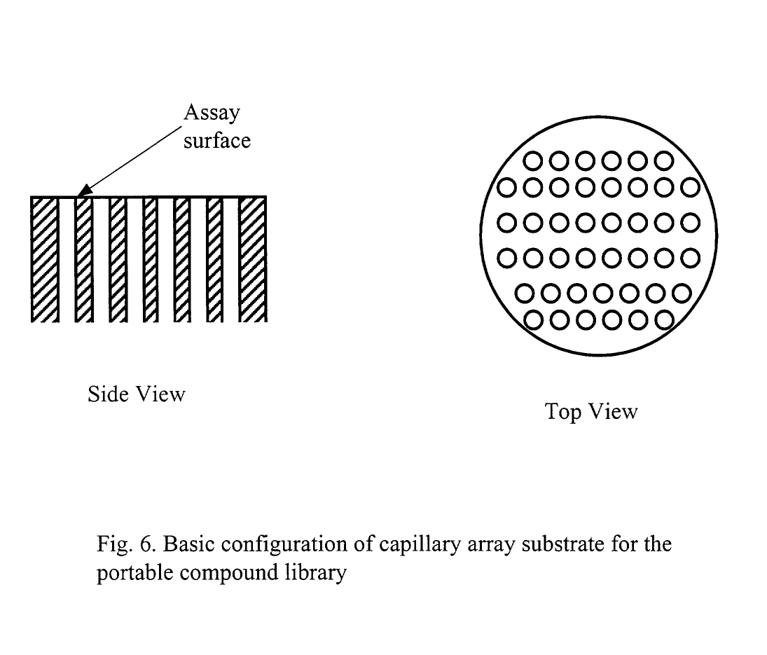

In one embodiment of the invention, the capillary array compound library is a portable medium that provides the means to facilitate compound storage, reagent metering, mixing and readout. As illustrated in FIG. 6, the basic configuration of the capillary array compound library comprises an array of assaying sites that are in fluidic connection to a common surface, which is termed the “assay surface”. Each assaying site may have at least one inner space capable of storing a compound and at least one other space for mixing reagents. The compound storage chamber is in fluidic connection with the reagent mixing space, and both are connected to the assay surface. Different compounds are held within the individual compound storage spaces. Additional reagents that are common to all assay sites are introduced to the assay surface and drawn into each assaying site which may have built-in fluidic features to perform or assist additional assaying functions such as volume metering, mixing and readout.

The number of assaying sites in the array directly relates to the number of screening assays to be performed in parallel, which is preferably more than 100, preferably more than 500, more than 1000, more than 5000, more preferably more than 10,000, more than 100,000 or more than 1,000,000. One or multiple such capillary arrays may be used to hold an entire compound library. The assaying sites are grouped on the assay surface at a spatial density of at least 40 per square centimeter, preferably more than 200 per square centimeter, more preferably more than 400 per square centimeter, more preferably more than 1,000 per square centimeter, or more than 4,000 per square centimeter, more preferably more than 10,000 per square centimeter, more than 40,000 per square centimeter, or more than 100,000 per square centimeter. The compound storage space at each assay site preferably holds fluid at a volume of no more than 100 microliters, preferably no more than 10 microliters, more preferably no more than 1 microliter, more preferably still no more than 100 nanoliters, preferably no more than 10 nanoliters, more preferably no more than 1 nanoliter, more preferably no more than 100 picoliters, more preferably no more than 10 picoliters, and more preferably still no more than 1 picoliter. The reagent mixing space preferably has a fluid holding capacity of no more than 10 microliters, preferably no more than 1 microliter, more preferably no more than 100 nanoliters, more preferably no more than 10 nanoliters, more preferably no more than 1 nanoliter, and more preferably still no more than 100 picoliters. For single-use compound libraries, the volume ratio of reagent mixing space over compound storage space is preferably greater than 10, preferably greater than 50, more preferably greater than 100 for HTS applications such as enzymatic assays. This volume ratio may be greater than 100, preferably greater than 500, and more preferably greater than 1,000 for HTS applications such as cell based assays. For other applications, such as a protein array or PCR, this volume ratio can be as small as 5 or even 2. It is desirable, for compound storage space in particular, to have a large volume (cubic micron) to exit opening (micron) ratio in order to reduce evaporation and potential contamination. In order to define this ratio as a pure number, the “volume” of an opening in this context is defined as the total volume of one or multiple largest possible spheres that can pass through the opening simultaneously. That is, the “volume” of an opening is the volume of a single sphere that can fit into the opening. If the channel has a circular cross-sectional area, the diameter of the sphere is equal to the diameter of the channel. The volume to opening ratio of the compound storage space is at least 2, preferably at least 10, more preferably at least 40, or more preferably at least 100, or at least 200. The volume to opening ratio of the reagent mixing space is at least 1, preferably at least 2, more preferably at least 5, or at least 10. The length to diameter ratio (“aspect ratio”) of the compound storage space is preferably no less than 10, 20, 50, or more preferably no less than 100, 200, or 500.

In one particular embodiment, an assay site comprises at least one hole substantially perpendicular to the assay surface. The internal structure of the hole comprises multiple interconnected chambers or wells or a combination of wells and chambers. In a further specific embodiment of the hole configuration, the assay hole is a through hole that has a second exit that may be on the same assay surface or on a second surface that is substantial parallel to the assay surface. The capillary array may be made of any suitable material such as glass, silicon, polymer, ceramic or suitable metal.

Formats

The capillary array compound library can take a number of physical formats. The formats described in this section are for illustrative purposes only and not exhaustive, and one skilled in the art may fabricate any number of configurations which are within the invention as described herein.

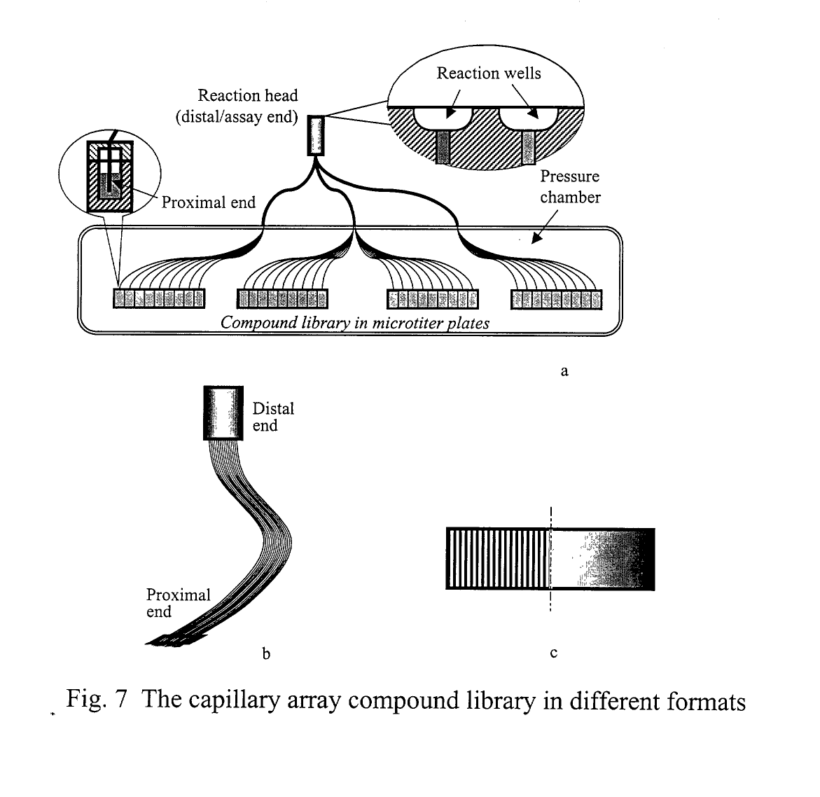

In a first configuration, referred to as a “branch” format, as shown in FIG. 7A, through holes are the channels of individual capillaries. The length of the capillary can range from about 100 meters to about 0.5 meter and the outer diameter of the capillary can range from about 2 mm to about 10 μm. For each capillary, a proximal end is inserted into a liquid reservoir (such as a well in a standard microtiter plate) while the distal end is bundled together with that of many other capillaries and formed into a solidified piece. In short, the capillary tube bundle in the loading station presented above is used directly for assaying. Additional features can be fabricated on the facet of bundled ends to facilitate reagent metering and mixing, as described in later sections.

A second configuration is referred to as a “bundle” format, as shown in FIG. 7B. The through holes are channels of individual capillaries which have outer diameters of about 2 mm to about 10 μm for instance. A large number of capillaries are bundled along the entire length from a proximal loading end to a distal reaction head end, either loosely or as a solidified unit. The diameter of the channel in the capillary is sufficiently small and the inner surface of the channel is sufficiently hydrophilic that liquid probes are retained within the channel by capillary force. The length of a bundle can range from about 0.1 m to hundreds of meters.

The array in bundle format can be fabricated directly from an array in branch format after individual liquid probes are pumped into the capillaries. The loose end of each capillary in the array can be taken out of the probe reservoir that it is inserted into and grouped together to form a capillary bundle that is bundled along its entire length. Liquid probes are stored within the cavities of capillaries and the stored volume is determined by the length of the capillary bundle and the inner diameter of the cavity. For example, a bundle of 1 m in length with a cavity diameter of 20 μm can store 0.3 μl probe liquid, sufficient for hundreds of experiments.

In a third configuration, referred to as a “chip” format, as shown in FIG. 7C, all through holes are formed in a solid piece, which takes a chip shape having a top 680 and a bottom 690 surface where probe liquids may enter and exit the through holes. Similar to the previously described formats, the diameters of the holes are sufficiently small and inner surfaces of the holes are sufficiently hydrophilic such that liquid probes are retained within the channel by capillary force. The thickness of the chip 692, and hence the length of the through holes, can range from about 50 μm to several tens of centimeters, preferably ranging from 200 μm to 1 centimeter, more preferably 500 μm to 2 millimeters. The size of a chip can be as small as 1 mm×1 mm, as large as 130 mm×130 mm. The through hole pattern can be randomly or orderly distributed. In the case of orderly distributed hole pattern, the hole pattern matches that of the delivery head capillary, or, in another example, matches the well pattern of a microtiter plate (96, 384, 1536, 3072, or 6144 well). A chip with microtiter plate pattern can be used as a “compound library cover” for a microtiter plate. The size of the chip can range from 5,000 cm² to 0.01 cm², or preferably from 1,000 cm² to 0.1 cm², or more preferably from 100 cm² to 1 cm². The array of assaying sites on the assaying surface has a spatial pitch ranging from 10 mm to 1 μm, or preferably 1 mm to 10 μm, more preferably 500 μm to 50 μm. The cross-section of the through hole may be circular or any other shape. Further, it may have the same shape and dimension along its length, or more preferably, it is structured to provide additional assaying functions as described in detail later. The through hole structure may have branches or junctions that involve multiple paths. In most cases, the through hole has its second opening on a second surface that is substantially parallel to the first surface, where the first opening of the through hole exits. It is also possible that the second opening of the through hole exits on the same surface as the first one. The diameter of the through hole ranges from 10 mm to 0.1 μm, or preferably from 1 mm to 1 μm, more preferably from 400 μm to 10 μm.

The capillary array chip can be fabricated in many different ways. It may be assembled from bundling ready-made individual long capillary tubes through out the entire length. The bundling can be achieved through epoxy or fusion bonding, for instance. The long bundle is then cut to a desired length. This method may be used to make a capillary bundle that has a hole pattern identical to the hole pattern of the capillary array chip. A capillary array bundle formed from an ordered array of capillaries is fused along its length such that multiple chips can be cut from the fused portion of the bundle. Once a number of chips are cut, a fused portion remains attached to the bundle and is is used for fluid delivery to the chips made from the bundle. This assures that the through hole pattern in the face of the capillary bundle is identical to the hole pattern in the chips cut from the bundle.

A second way to form a capillary chip is to bundle large preform tubes together and extrude the preform bundle into a long solid capillary bundle, then cut the bundle to form chips of desired length(s). A third way to form a capillary chip is to mold a large preform having an array of through holes using a suitable powder mixture, usually made of a ceramic or metal oxide. The powder is solidified through heat fusion, then extruded to reduce to the capillary pitch and finally cut to desired length(s) to form the capillary chip(s). The fourth way of forming a capillary chip is to start with a solid chip substrate made of silicon, glass, plastic, ceramics, metal oxide, metal or other suitable materials. Through holes are fabricated in the substrate using available micromachining technologies, which are widely used for microelectromechanical systems (MEMS) applications and include etching, especially deep reactive ion etch (DRIE), laser drilling, mechanical drilling, ultrasonic drilling, sand blast drilling, micro-molding, LIGA (lithography, electroforming, and molding), electric plating and wafer bonding. One additional way to form a capillary array chip used to form a capillary array compound library is to form individual features in separate slides or substrates, the join or fuse the separate pieces together to form the chip. For example, a reaction chamber may be formed in silicon substrate #1 by etching the substrate using MEMS fabrication technology, capillary through holes may be formed in two separate silicon subsgtrates #2 and #3 by etching them, narrower channels that act as flow restrictors between capilllary through holes may be formed in silicon substrate #4, and the substrates may be stacked in the order substrate #1/substrate #2/substrate #4/and substrate #3 and then fusion bonded together in an oven to form the capillary array chip or capillary array compound library.

Most fabrication methods for chip format capillary array are to make a chip substrate with empty through holes first, then use a dedicated loading station as described above, for example, to load compound solution into the holes. In an alternative method, a compound solution is first loaded into the channel of a very long, stand-alone capillary by pressure. Then the solution can be dried in the capillary. Alternatively, the capillary can also be frozen to fix the compound in place in the capillary. Next, many such capillaries filled with different compounds are bundled together using various bonding methods including gluing, diffusion bonding, soldering, or other method known to one of ordinary skill. Finally, the very long bundle can be cut to length as required. The cutting can be carried out using various devices, which include a diamond saw (wire and disk), laser, water jet, plasma beam and other known cutting system.

Internal Structures of Through Holes and Their Functions

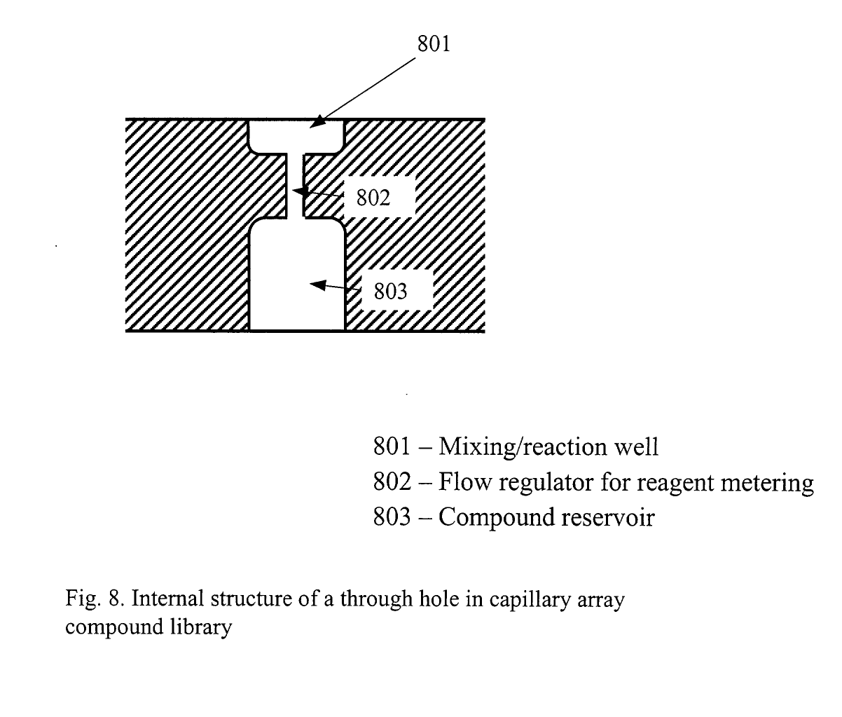

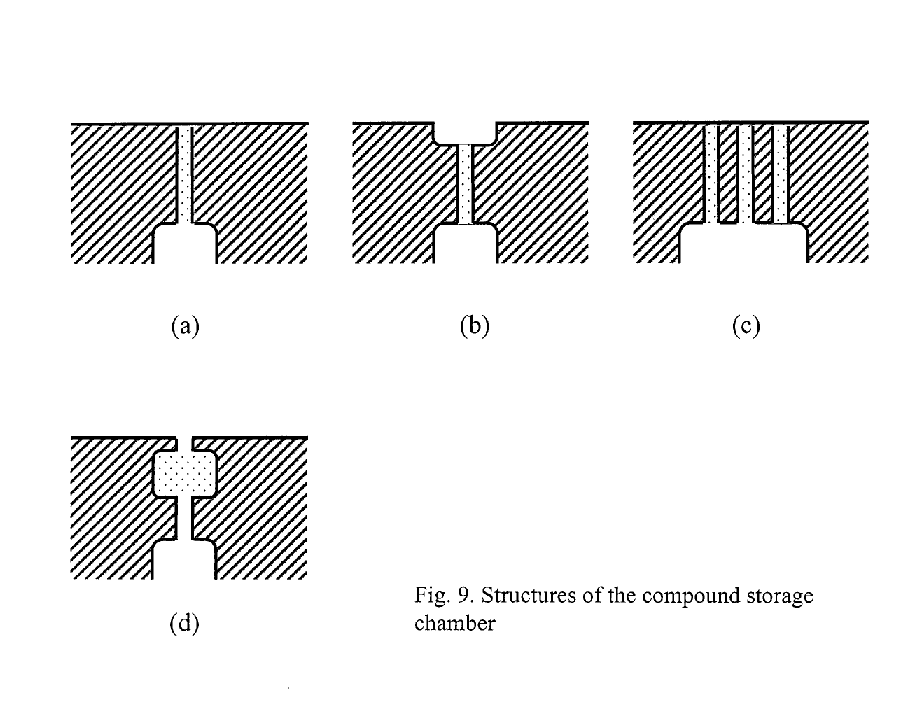

Preferably a capillary array compound library comprises an array of through holes. Each through hole may provide a means to store, meter and mix reagents used for the assay and to assist readout results. FIG. 8 illustrates one embodiment of the internal structure of a through hole. This is a typical structure which generally comprises a reservoir for compound storage, a chamber for reagent mixing and reaction, and additional features on the assay surface that localize liquid to particular areas to prevent cross-contamination during compound and reagent loading. Other functions may also be integrated in the through hole structure which enables precision metering of reagents, reduces evaporation and assists optical detection, respectively. These structural features and their functions are described in detail below.

Compound Storage Chamber

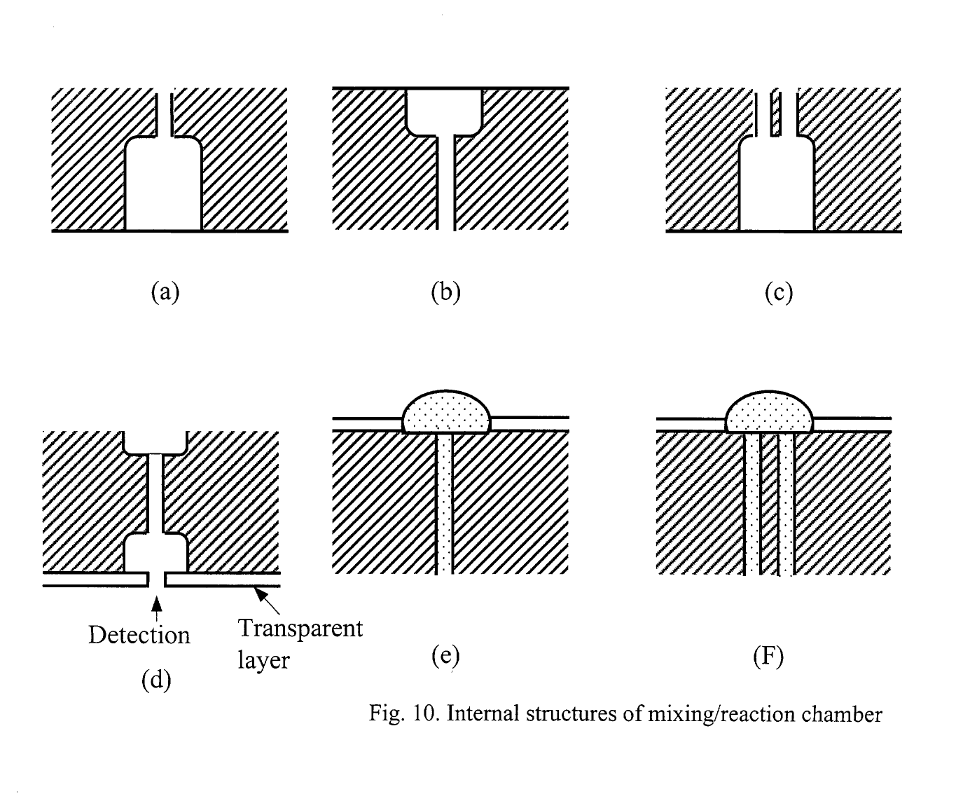

Compound storage in a miniature and portable form is the basic function of the capillary array compound library. Preferably, each compound is in pure DMSO (dimethylsulfoxide) solution or other polar solvent and is stored in a chamber along the through hole (FIGS. 9a, b), or, in some cases, in multiple through holes (FIG. 9c). The through hole structure is ideally suited to store solutions in very small volumes as called for in HTS applications. As the evaporation rate is directly proportional to the surface area exposed to air, evaporation can be minimized by using tubes with small diameters or small openings for compound storage (FIG. 9d). Evaporation may further be minimized by sealing or covering the capillary ends using e.g. a polymer or metallic film adhered to the edges of the surface of the substrate. Preferably, the film is hydrophobic to prevent the film from removing any liquid from a through hole when the film is removed. Inert gas may be used to extend compound storage shelf life as well.

The inner volume of the storage chamber can be designed to hold sufficient compound volume for single or multiple uses. In certain applications, the compound solution may be dried after the compound is loaded into through holes using the loading station. The dried compound powder will reside inside the storage chamber, preferably attached to the inner wall.

A re-dissolving stage is carried out using pure DMSO or other polar solvent in the screening station after the capillary array compound library is shipped to the users. This will be discussed in a later section.

“Reagent Mixing”/Reaction Chamber

In a typical enzymatic assay concerned with HTS, reagents include three different solutions, i.e. compound, enzyme and substrate. These reagents have to be mixed thoroughly and incubate for a certain period of time. The invented library provides a structure for the mixing of reagents required in an assay. This structure can be a chamber in the through hole, which is similar to the compound storage chamber but usually much larger in volume and dimensions, as shown in FIGS. 10a and 10b. The mixing chamber may link to multiple parallel chambers to receive different reagents (FIG. 10c). A cover with a very small opening is integrated to the mixing/reaction chamber in the design shown in FIG. 10d to reduce evaporation during incubation. This cover is preferably transparent to allow optical reading through the cover.

A well on chamber as discussed herein may be either a physical well, such as a depression in a surface, or a virtual well. FIGS. 10e, f show an alternative “virtual well” design for the mixing/reaction chamber, which comprises a hydrophilic patch around the entrance of a through hole. The patch is surrounded by a hydrophobic region. Fluid can be held within the boundary of the patch by surface tension force.

The invention may also provide structural features that enhance mixing. As illustrated in FIGS. 10a to 10f, the mixing chamber has a much larger cross-section in comparison with that of the path between the reagent reservoir and the mixing/reaction chamber. A micro vortex can be generated when the reagent flows into the mixing chamber, which greatly enhances mixing, by moving the fluid rapidly through the capillary and into the reaction chamber. Additional microfluid features can be built at the entrance to the mixing chamber to further enhance the mixing. These include micro-comb or micro-hive structures that split flow into many branches resulting in enhanced diffusion and creation of micro-vortexes.

Metering

In most modern scientific studies involving biological or chemical reactions, volumes of the reagent fluids that take part in the reaction have to be very precise in order to obtain the desired result. Currently, control of reagent volume is usually achieved by using a dedicated device that meters the fluid volume and then dispenses the measured volume into a container for mixing and reaction.

There is a great need in reducing the volume of reagent fluids used in a screening experiment. This is especially true in the area of high throughput screening (HTS). This is because most compounds used in HTS are expensive and some are precious and purified from rare natural substances. In addition, preparing a large quantity of sample is time consuming, costly and sometimes impossible. Currently, a state-of-the-art HTS system requires dispensing volumes of compounds on the order of tens of nanoliters with standard deviation (“CV”) less than 10%. Such a requirement for precision is very challenging using existing metering and dispensing technologies. At such a small volume, the amount of residual fluid left on the tip of the dispenser becomes a significant factor affecting the dispensing CV as it is very difficult to eliminate the residual or make the residual volume consistent. This has become a major bottleneck in further reducing the reagent consumption.

There are a number of ways to meter reagent volume on the destination container. One method is to dispense an approximate amount of required volume, then precisely measure the actual volume using visual or other means and eventually factoring the actual volume into the final result mathematically. This method requires direct measurement and mathematical manipulation of data to derive the information desired.

This portion of the invention provides a novel concept that integrates containing, metering and mixing functionalities into a single platform, which reduces or eliminates the fluid volume error caused by reagent dispensing as described above.

The reason why dispensing becomes a major source of volume error is due to the fact that reagents are dispensed after they are metered in a dedicated equipment. This invention proposes to eliminate such error sources by a completely new approach: metering at the destination after dispensing the reagent. This method and system are applicable to many microfluidic systems in which accurate droplet metering is required.

One specific embodiment of this invention is to design the destination container so that it not only is used as a container for reagent mixing and reaction but also facilitates additional functions such as reagent metering and readout. In this design, excess reagent is dispensed to the assay surface of the library or destination container, then geometric or other fluidic constraints retain a desired volume on the surface or in a designated chamber. Excess fluid is then removed from the destination container. Three different embodiments of this configuration are presented below:

Metering with Surface Tension Patch

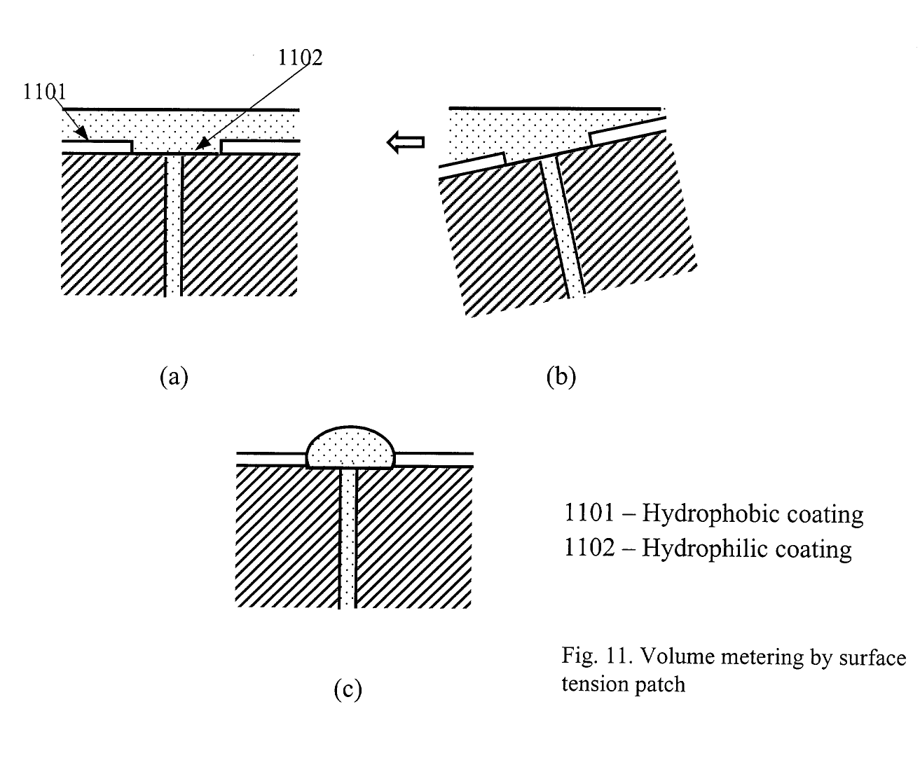

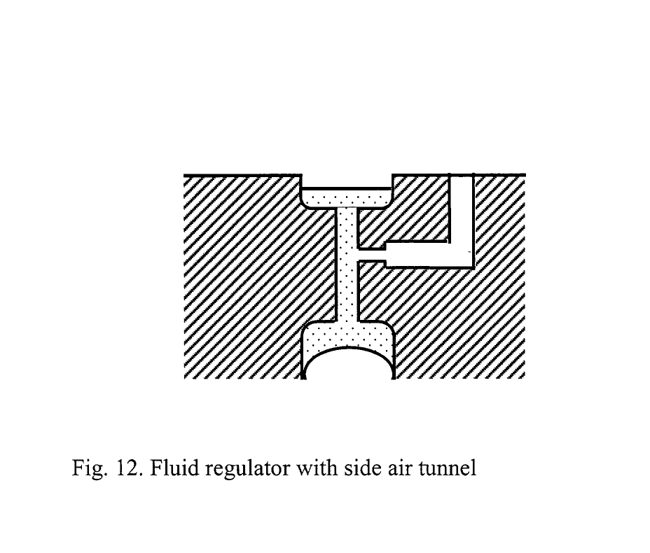

As illustrated in FIG. 11, a hydrophilic patch 1102 is surrounded by a hydrophobic area 1101. As described in the above section, this configuration forms a “virtual well” and is capable of holding a certain amount of fluid. The fluid volume that can be held by the patch is determined by the size of the patch and fluid contact angle of the hydrophobic area surrounding the patch (11c). Metering is achieved by applying abundant fluid to the patch (11a) and removing the excess fluid by various methods, which include tilting the surface at an angle sufficient to allow excess reagent to run off the surface (11b), centrifuging or applying a vacuum of a suitable strength (11b).

In this instance, the liquid to be dispensed at through holes of a library has a surface tension that produces a droplet of given volume in the hydrophilic region in which the droplet forms. The surface of the droplet at the hydrophobic/hydrophilic interface on the surface of the library has a certain contact angle that depends on the surface tension of the liquid being dispensed. The size of the droplet is thus a function of the size of the hydrophilic area (if the liquid being dispensed is polar) and the contact angle of the droplet's surface. Thus, there are two ways to meter appropriate volumes of liquid. One is to provide a hydrophilic patch of desired size for a liquid of given surface tension, and the other is to adjust the surface tension of the liquid to form a droplet of the desired volume in a hydrophilic patch having a given or known size. Surface tension of liquids may be adjusted by means known to those of ordinary skill in the art, and these include adding salts such as sodium chloride and potassium chloride, and detergents such as sodium dodecylsulfate (SDS), sodium lauryl sulfate, and sodium laureth sulfate (SLS).

Another design of this invention involves creating microgrooves circling or otherwise surrounding the immediate opening of through holes. Such microgrooves retain solution by capillary action. Preferably, the width of the grooves is no more than 100 microns, more preferably no more than 20 microns, and even more preferably no more than 10 microns. Preferably, the depth of the grooves is no more than 50 microns, more preferably no more than 15 microns, and even more preferably no more than 5 microns.

Metering with a Flow Regulator

In one embodiment of this invention, a flow regulator is provided between the mixing/reaction chamber and the reagent reservoir. The volume of reagent delivered to the mixing chamber can be controlled by external fluid pressure and its application duration. This regulator can be simply one or multiple narrow paths linking a mixing chamber and the reagent reservoir, as shown in FIG. 8. The narrower the path, the more control there is over the flow. In the structure shown in FIG. 8, the mixing/reaction chamber maintains a fluid connection with the reagent reservoir. FIG. 12 shows a more sophisticated regulator structure, which provides a side air tunnel linking the air above the mixing chamber to the narrow path. At the end of reagent delivery, the pressure on the reservoir side will decrease and draw in air from the side tunnel, which forms an air bubble to isolate the reagent in the reservoir from the fluid in the mixing chamber.

Metering with Through Holes

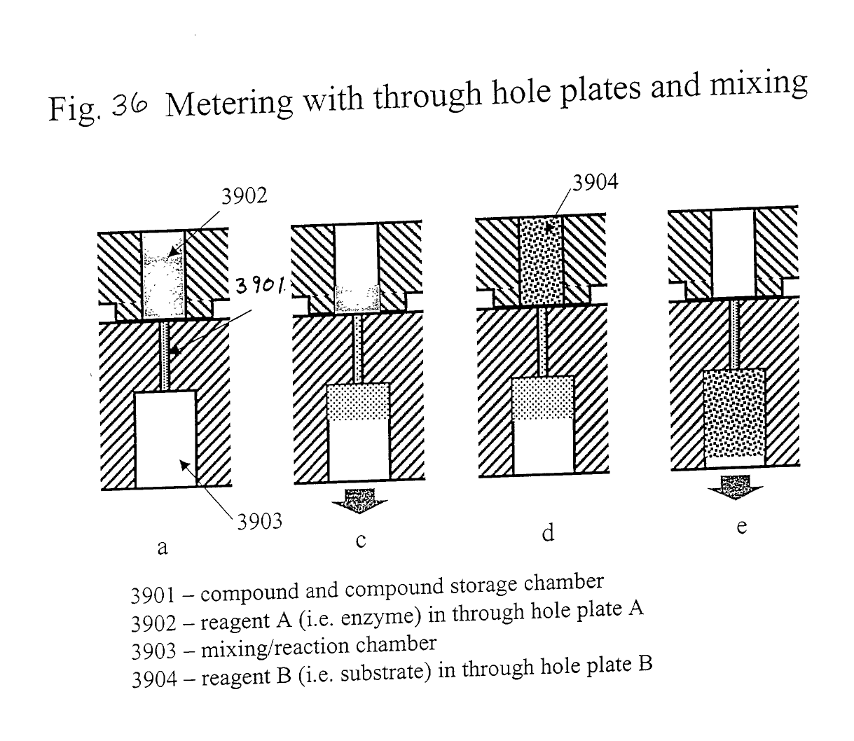

This invention also uses the inner space of a through hole to meter reagents. The inner surface of the through hole is preferably made hydrophilic. When a fluid is present at the entrance of the hole, the capillary force will thus draw fluid into the hole. If an excessive amount of fluid is present, the entire inner space of the hole will be filled. By removing the rest of the fluid outside the hole, the fluid volume is metered to be equal to the volume of the inner space of the through hole. Different reagents can be metered with separate through hole plates. To mix these reagents, through holes on different substrates can be aligned to establish a fluid link to a larger mixing chamber. Pressure will be provided to drive fluids through connecting through holes into the mixing chamber. This is illustrated in detail in FIG. 36. Preferably, the diameter of the through hole for holding reagents is 50% or more larger than the diameter of the compound storage capillary, more preferably 100% or more larger, and even more preferably 300% or more larger. Preferably, the ratio of the space of each through hole in the reagent plate to the space for holding each compound in the capillary compound library is more than 10, more than 50, more than 100, or more than 1000. Proper level of compound dilution can be achieved with such ratios.

Metering with Interconnected Chambers

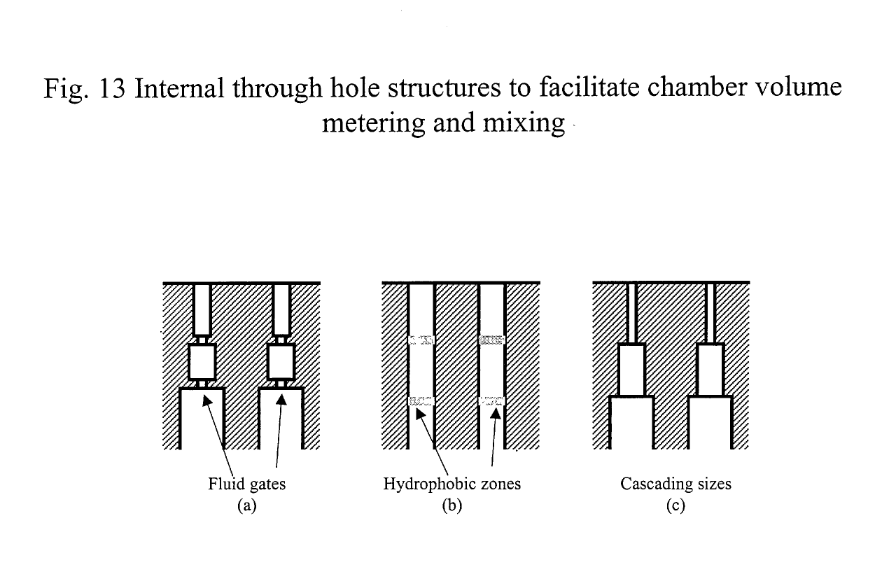

This embodiment of the invention uses a chamber in the through hole to meter reagents. The inner surface of the chamber is made hydrophilic and is separated from other portions of the through hole by a fluid barrier, which prevents fluid from crossing when the pressure differential is less than a certain “bursting pressure”. Such barrier may be a short narrow opening as shown in FIG. 13a or a short hydrophobic zone (FIG. 13b). Alternatively, it can be an interface from a smaller to a larger chamber (FIG. 13c) or a combination of any of these. One method for constructing such internal structure is to build each chamber on a separate wafer using existing micro-fabrication methods such as deep reactive ion etching, micro molding, electro plating or chemical vapor deposition and then bonding the multiple wafers together. The chamber has a sufficiently small cross-sectional area that fluid is drawn into the chamber by capillary force when fluid is present at the entrance to the chamber. When the fluid fills the chamber to the fluid barrier, capillary force prevents the fluid from breaching the barrier, thus confining a definite amount of fluid to the chamber. After the chamber is filled, excess fluid can be removed from the top surface of the substrate by one or a combination of the following means: 1) blotting, 2) drawing excess fluid from the surface using a vacuum pressure that is less than the “bursting pressure”, 3) capillary force using another dry capillary array placed on the wetted surface of the first capillary array to draw excess fluid from the surface of the first capillary array using capillary force; 4) wiping, and 5) air knife blowing. In method 4, the pore size or pore cross-sectional area of the dry capillary array that is used to remove the excess liquid should in general be larger than the pore size or pore cross-sectional area of the capillaries in the first array in order to avoid withdrawing liquid from the designated reagent chamber of the first array. In this way, liquid inside the through hole of the first capillary array will not be removed.

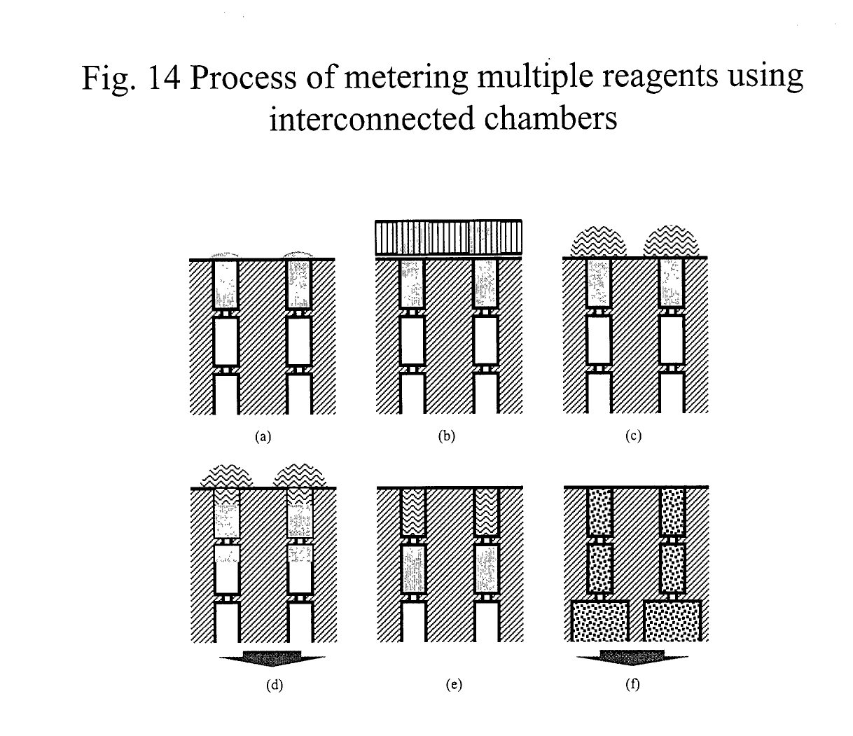

If an assay requires the mixing of multiple different reagents, multiple interconnected chambers can be used to meter every reagent, as illustrated in FIG. 14. The first reagent applied to the substrate is drawn into the first chamber of the through hole and held there by capillary force. The fluid is isolated from the second chamber in the hole due to the “bursting pressure” created at the interconnecting region between chambers, as shown in FIG. 14a. After removing excess first reagent, the second reagent can be applied to the top surface using a capillary bundle fluid delivery system, as shown in FIGS. 14b and 14c. Then a short pulse of driving pressure can be applied, which can either be negative pressure applied to the bottom side of the substrate to draw liquid in or positive pressure applied to the top side to push liquid in. In either case, the driving pressure is greater than the “bursting pressure” of the fluidic barrier between the first and second chamber. This results in the fluid in the first chamber bursting into the second chamber. Once the barrier is burst, capillary force takes over and draws liquid into the second chamber. Because the first and second chambers are connected, the second reagent on the top surface also is drawn into the through hole, as shown in FIG. 14d. Excess second reagent on the top surface can be removed and the container is ready for the loading of subsequent reagents (FIG. 14e). This process can be repeated as many times as the number of chambers in the through hole (FIG. 14f).

After all reagents are loaded into the through hole, mixing can be achieved by diffusion or alternatively, all reagents in different chambers of a through hole can be pumped into a larger chamber at the end of the through hole, where it will mix and incubate at a higher efficiency, as shown in FIG. 14f. The reaction results can be read from the through hole by optical or other means.

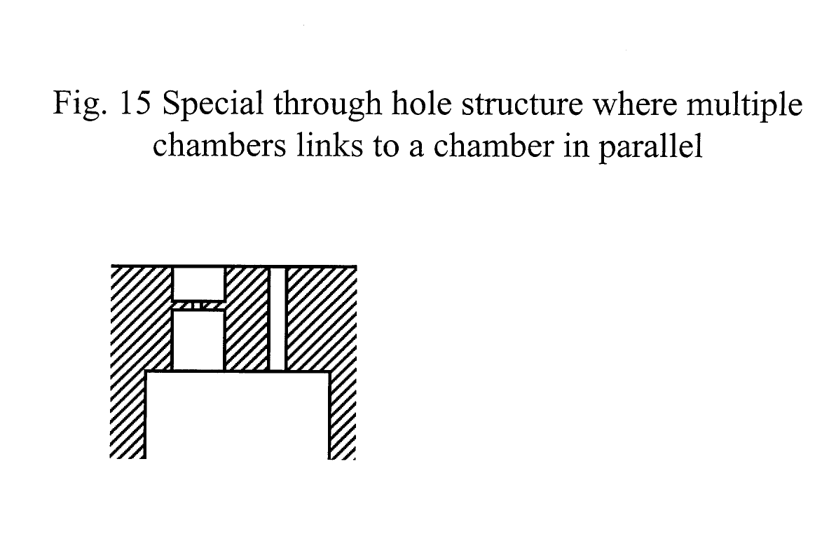

The structure and loading method described above is sequential for each container. FIG. 15 illustrates a different container structure, which comprises multiple chambers linked to a large mixing chamber in parallel (only two parallel chambers are shown in the figure). The different reagents can be loaded in parallel to different chambers of a container using e.g. a capillary fluid delivery system as described previously. The total required number of such fluid loading chambers in a container in the vast majority of applications is not very large because many reagents can be pre-mixed in bulk prior to delivery to the substrate.

Excess Fluid Removal

In many fluid metering methods described above, fluid is delivered to the destination container, which is metered by an intrinsic reagent reservoir or chamber and any excess fluid outside the reservoir is removed. This invention provides a number of methods to remove excess fluids. These methods can be used alone or in combination.

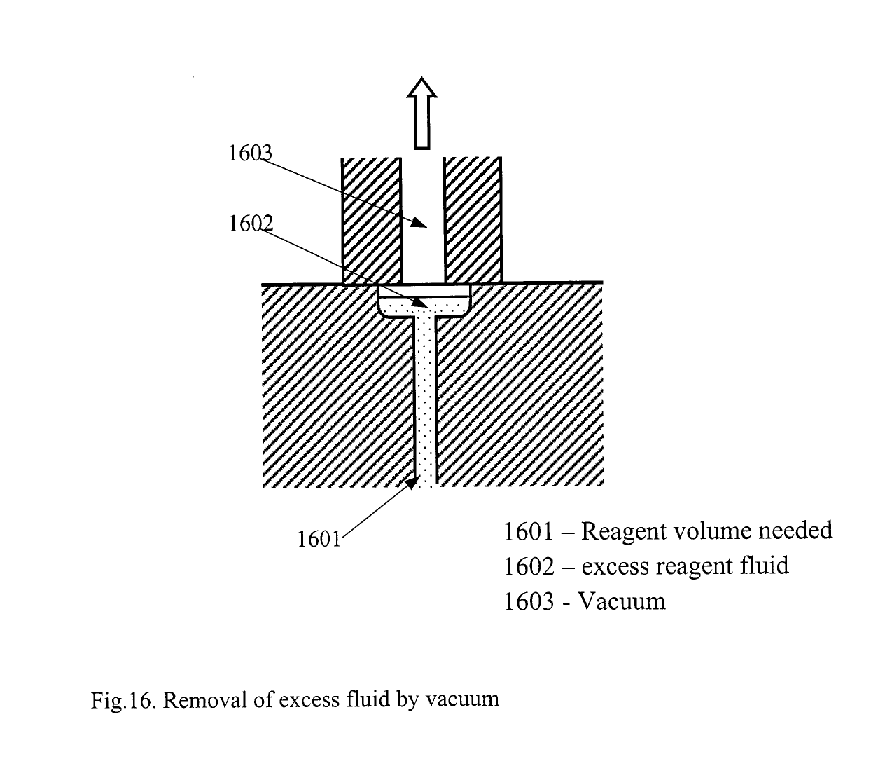

The first method is to use vacuum force. The vacuum pressure has applied is less than the “bursting pressure” of the reservoir entrance that holds the metered fluid. As illustrated in FIG. 16, the substrate has a physical well with a much larger cross-section than the entrance of the metered fluid reservoir has. The capillary force in the reservoir is much greater than that in the well. A vacuum force selected to be smaller than the capillary force in the reservoir but larger than that in the well can be applied to remove excess fluid from the well while leaving the metered fluid in the reservoir intact.

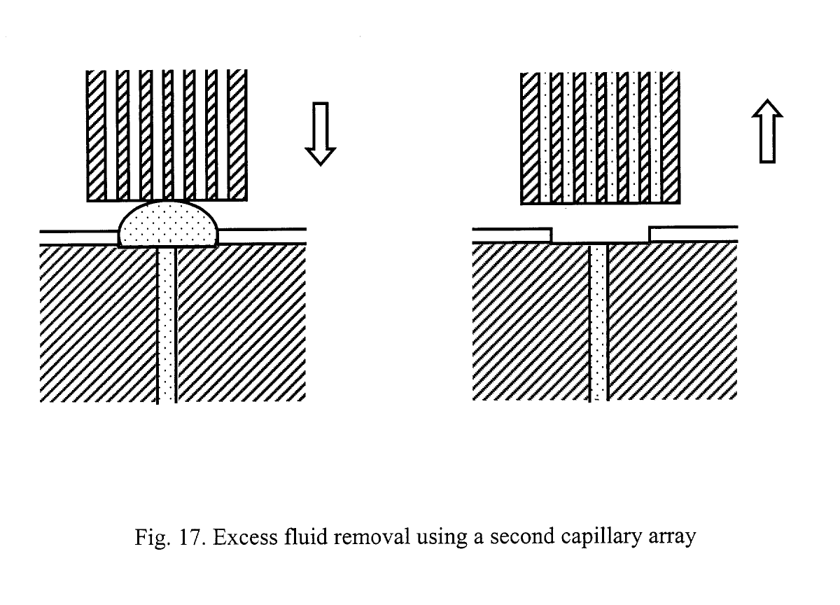

The second method is to blot the excess fluid with a suitable porous material, which can be a tissue or another capillary array for example. A tissue with suitable porous fiber composition can soak out the excess fluid positioned outside the metered reagent reservoir without removing liquid inside the reservoir. As illustrated in FIG. 17, a capillary array second with or without a matching through hole pattern whose capillaries have a capillary force slightly below that in the reservoir can be brought into contact with the excess fluid, which will draw the excess fluid outside the reservoir into its capillaries without removing fluid inside it.

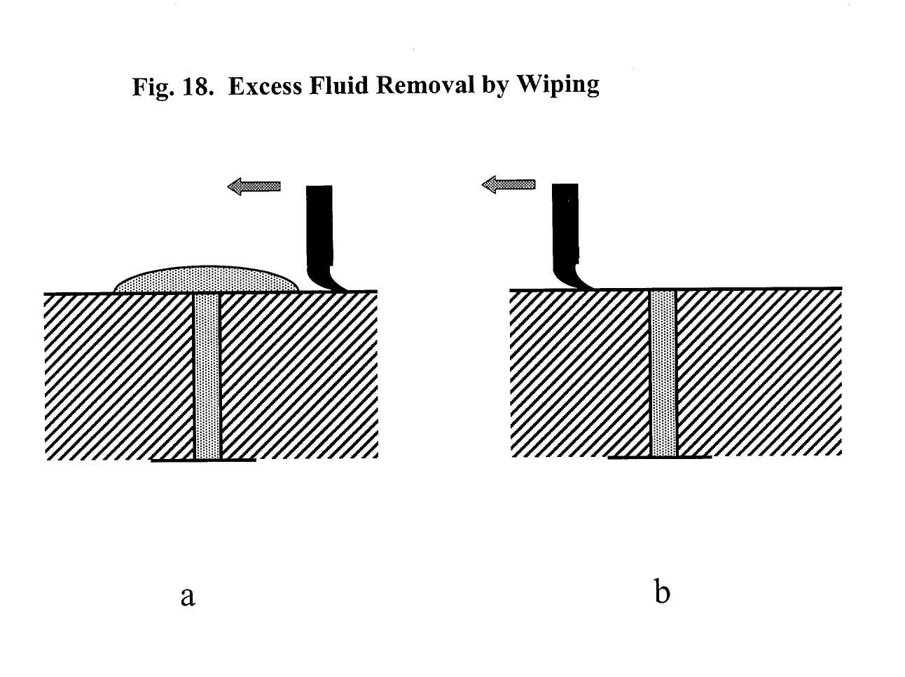

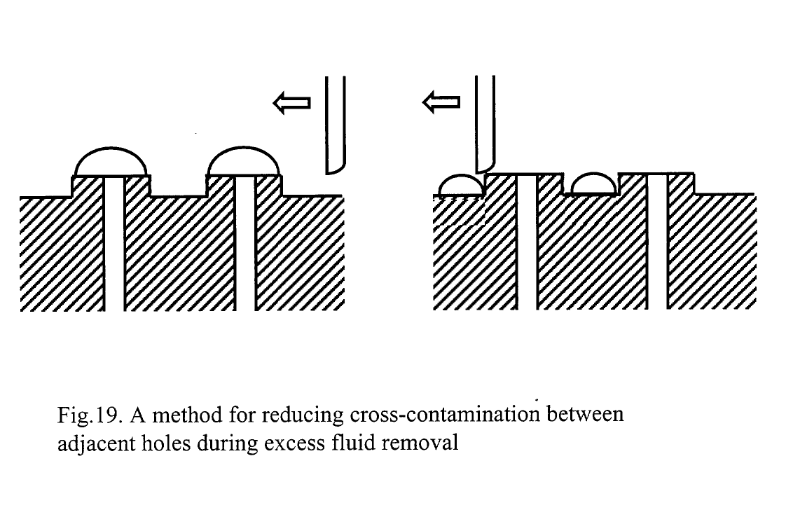

The third method is to mechanically wipe away excess fluid using a precision edge, as illustrated in FIG. 18. This method is suitable for structures where the excess fluid resides on a flat surface. The edge can be made of soft and non-porous material such as rubber or soft and porous material like a sponge. In this case, wiping and blotting is combined to remove the excess fluid. The edge can instead be an “air knife” that blows away excess fluid. This method may potentially introduce fluid cross-talk between different through holes if the pressure used is too high. This is not an issue if all fluid at the entrances to different fluid reservoirs are the same fluid. This invention also provides means to reduce fluid cross contamination. As illustrated in FIG. 19, each reservoir entrance is isolated geometrically by fabricating an island around it. Excess fluid falls into the gaps between these islands in a wiping action, thus reducing the chance of cross-talk between different reservoirs.

Islands may be formed by molding them into the surface during fabrication of the compound library substrate, for instance. Alternatively, islands may be formed by placing a patterned photoresist on areas that are to become islands and etching surface that is not protected by the photoresist. Likewise, the capillaries may be bound by an adhesive that has a substantially different etch rate from the capillaries, and the adhesive may be etched to remove a small amount, leaving capillaries standing slightly proud of the surface. This latter method obviates the need for masking the surface. Etchants include H₂SO₄, nanostripe, etc.

Optical Signal Readout

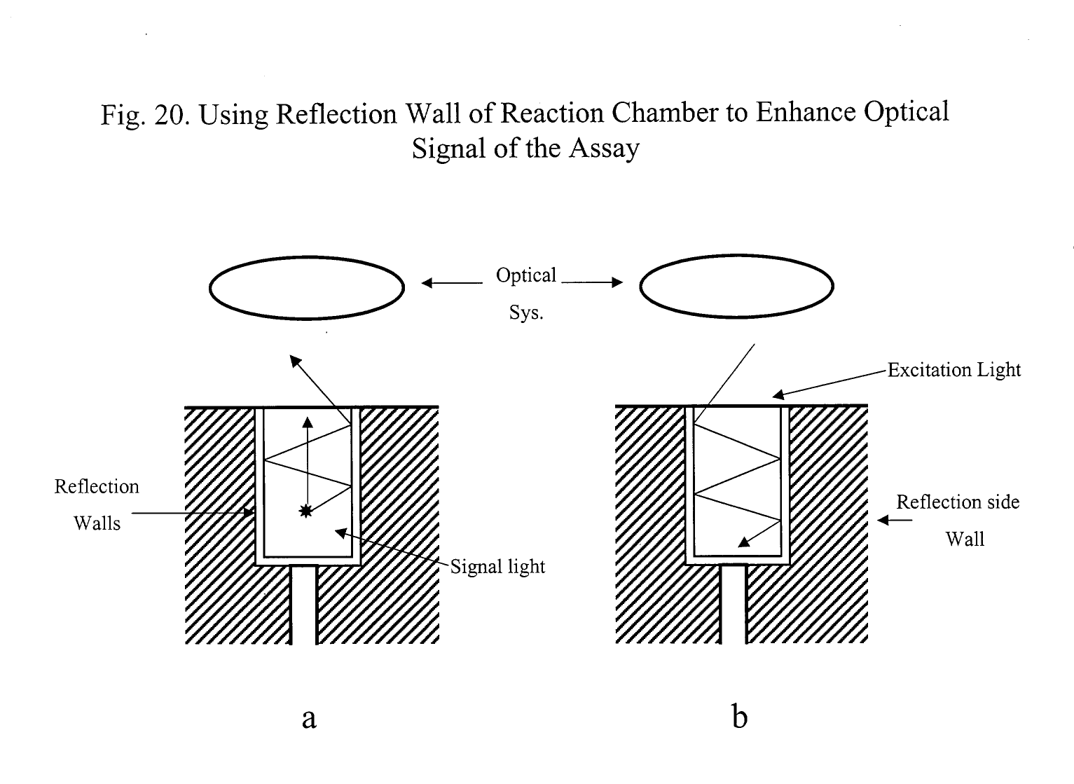

In a preferred embodiment, the invention provides features in individual through holes of the capillary array to assist readout of optical signals generated during the assay. FIG. 20 illustrates one embodiment of the design, where the inner wall of the mixing/reaction chamber of the capillary array is made highly reflective. This metal coating has two benefits: first, in a miniaturized structure, the wall between different reaction chambers may become too thin to efficiently block light from adjacent walls of wells. This may cause signal cross-talk and may reduce signal to noise ratio of the detection. A highly reflective layer is very efficient in attenuating light transmission between adjacent mixing/reaction chambers or through holes. Second, the metal coating enables a large percentage of the signal light that hits the wall that would otherwise be lost from an uncoated chamber to be eventually collected by the detection optics by directing the light to the optics through multiple reflections between chamber walls, as illustrated in FIG. 20a. Third, in fluorescence assays, one way to enhance signal to noise ratio of the detection is to enhance the fluorescence emission while suppressing excitation light that may be collected by the detection optics. A reaction chamber designed for fluorescence assays is built with a highly reflective side-wall and a bottom with a high degree of absorption. A major part of the excitation light will also bounce many times between the walls of the chamber, which excites the fluorescent marker multiple times thus multiplying the strength of the fluorescence signal (20b). Once the excitation light hits the bottom of the reaction chamber, it will be largely absorbed and thus will not bounce back to the opening, thus avoiding its collection by the detection system. The reflective layer in the chamber can be fabricated by coating a metal layer, such as gold, aluminum or copper by vapor deposition or sputtering. The coating is preferably only as thick as is needed to coat the walls to provide a reflective surface. Alternatively, the entire structure of the chamber can be built with metal material using e.g. an electric plating technique commonly employed in microfabrication of MEMS devices. In this technique, a substrate surface is first coated with a conductive layer, such as gold, suing vapor deposition. Then, a layer of photoresist is added. A lithography process and etching are employed to open up locations where metal structure is needed. Metal, such as nickel or copper is deposited in these designated locations by an electro plating process. On the other hand, a “grass” like surface feature can be fabricated on the bottom of the reaction chamber to significantly increase the absorption. Such surface features can be achieved through high ion strength bombardment during dry etching.

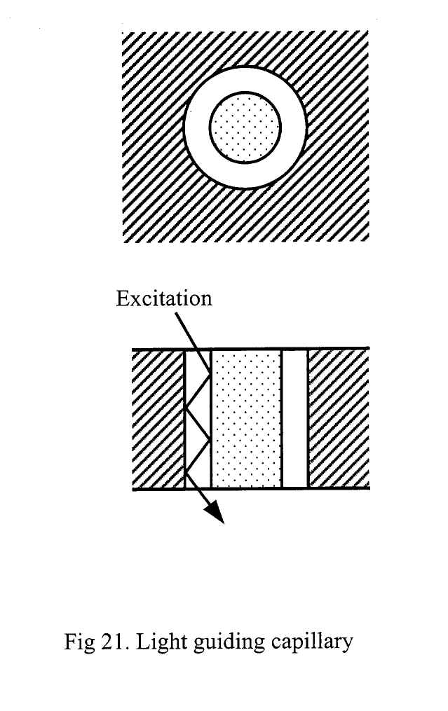

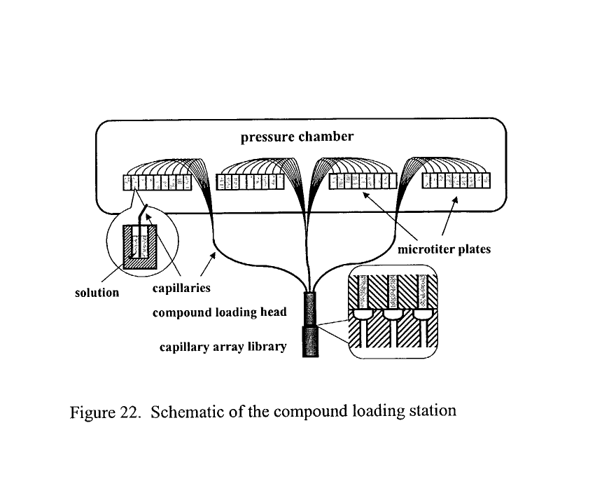

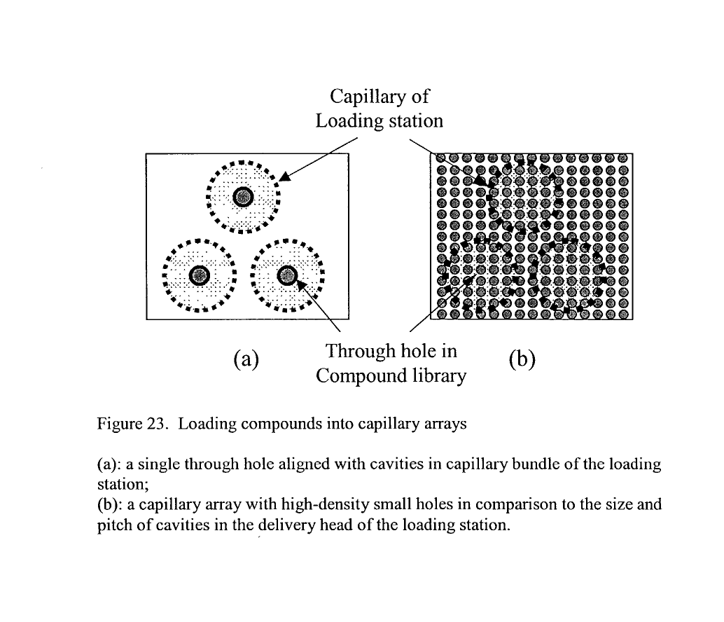

FIG. 21 illustrates another embodiment of the reaction chamber design that facilitates optical detection. In this design, a circular optical wave guide is built around the reaction chamber. The wave guide is formed by constructing a layer of optically transparent material with a higher refractive index than the adjacent regions. This layer can be made of pure silica, doped silica or suitable optical polymer. Such light guiding structure can be fabricated in a number of ways. In one embodiment, the light guiding layer is fabricated on the inner wall of a silica tube preform by either doping Germanium in the inner wall in a process termed MCVD (modified chemical vapor deposition) or doping fluorine on the outer wall using OCVD (outside chemical vapor deposition). The preform can be extruded into thin capillaries. A large number of such capillaries can be bonded together and cut to desired length to form a capillary array chip. Finally, this chip can be used as the capillary array compound library or may be bonded to a wafer containing other assay features to form the library. In another embodiment, the capillary array chip is prefabricated in silica or quartz. Ge or fluorine doping can be introduced to appropriate surface areas through ion assisted implantation. In fluorescent-based binding assays, the probe can be immobilized on the inner wall of the reaction chamber. The excitation light that enters the wave guide will generate an evanescent energy field along the inner wall of the reaction chamber. If the fluorescence labeled sample molecules bind the probe on the wall, they will be excited by the evanescent field and the signal light can be collected at either end of the wave guide. This configuration enables some very useful assays as described in a later section.

(b) Compound Loading Station

This invention provides a compound loading station that delivers compound fluids from a traditional storage medium to a capillary array compound library. The compound loading station is a system that is capable of injecting compound solutions from wells of microtiter plates where the compound solutions are originally stored into separate through holes of the capillary array.

As shown in FIG. 6, one embodiment of the compound loading station comprises a pressure chamber and capillary bundle. The fluids to be delivered are stored in individual reservoirs, which could be wells in standard microtiter plates. These reservoirs are placed inside the pressure chamber. One or multiple capillaries are inserted into each reservoir, which guide the fluid towards the distal end where all capillaries are bundled together. The fluids are driven from one end of the capillary to the other by one or a combination of the following mechanisms: pressure, or gravity, or capillary force, or electric field or magnetic field. The bundle holds the distal ends of capillaries in a specific spatial pitch and pattern. Such a sub-system is described in detail in U.S. patent application Ser. No. 09/791,994 which is capable of delivering a very large number of small quantities of different fluids in parallel.

During the compound loading process, the capillary array with empty through holes is placed against the facet of the loading head. In one specific design, as shown in FIG. 7a, the pitch and pattern of the capillaries at the loading head are the same as that of the through holes in the capillary array. This system can be made by forming a long bundle and cutting the bundle into two pieces of desired length to form the capillary array and capillary bundle of the compound loading station. A system which utilizes an array chip can be made by forming a long bundle and cutting the bundle into three pieces of desired length to form the capillary array, capillary bundle of the compound loading station, and array chip.

In another design, shown in FIG. 7b, the through holes have a much denser pitch than that of the capillary in the loading head. In both cases, a capillary in the loading head is aligned with one or a cluster of through holes in the capillary array. A positive pressure is applied in the pressure chamber to push the compound solution into the holes of the capillary array. Alternatively, a negative pressure is applied to the other end of the capillary array to suck the compounds into separated through holes in the capillary array.

After loading, the compound solutions can be sealed inside separate through holes by applying a cap on each side of the capillary array to prevent evaporation. The capillary array is then transported and distributed to the users as a miniature compound library without evaporation.

Instead of using dedicated loading stations described above, an alternate system can be used to make the capillary array-based compound library. A compound solution is first loaded into the channel of a very long, stand-alone capillary by pressure. Then the solution can be dried to enable the compound to solidify in the capillary. Alternatively, the capillary can also be frozen to fix the compound in place in the capillary. Next, many such capillaries filled with different compounds are bundled together using various bonding methods including gluing, diffusion bonding, soldering, or other method known to one of ordinary skill. Finally, the very long bundle can be cut to length as required. The cutting can be carried out using various devices, which include a diamond saw (wire and disk), laser, water jet, plasma beam and other known cutting system.

Currently, almost all compound libraries are stored in standard micro titer plates. Concentrated solutions are in “mother plates” for long-term storage. Periodically, compounds are diluted into “daughter plates” in central compound management facilities. In response to the request from HTS centers, compound solutions may be further diluted into “working plates” and transported to HTS centers in sealed packages.

The loading station in this invention provides means to accept and hold microtiter plates, means to accept and hold capillary arrays and means to interface between the two. FIG. 25B illustrates one embodiment of the loading station, where the interface between the micro titer plates and the miniature capillary array is provided by a bundle of capillary tubes that has two distinguishable ends. The capillary bundle for delivering a library of compounds can be designed as described in pending U.S. patent applications Ser. Nos. 09/791,944, and 09/791,998. At one end, the tubes are bundled together to form a matrix that is compatible with the array of microscopic reaction sites on the miniature capillary array. At the other end, the tubes are loose, and thus the tubes can be inserted into individual wells of the micro titer plates. The compound fluids are transported from the micro titer plates on to the miniature capillary array in parallel through the tubes.

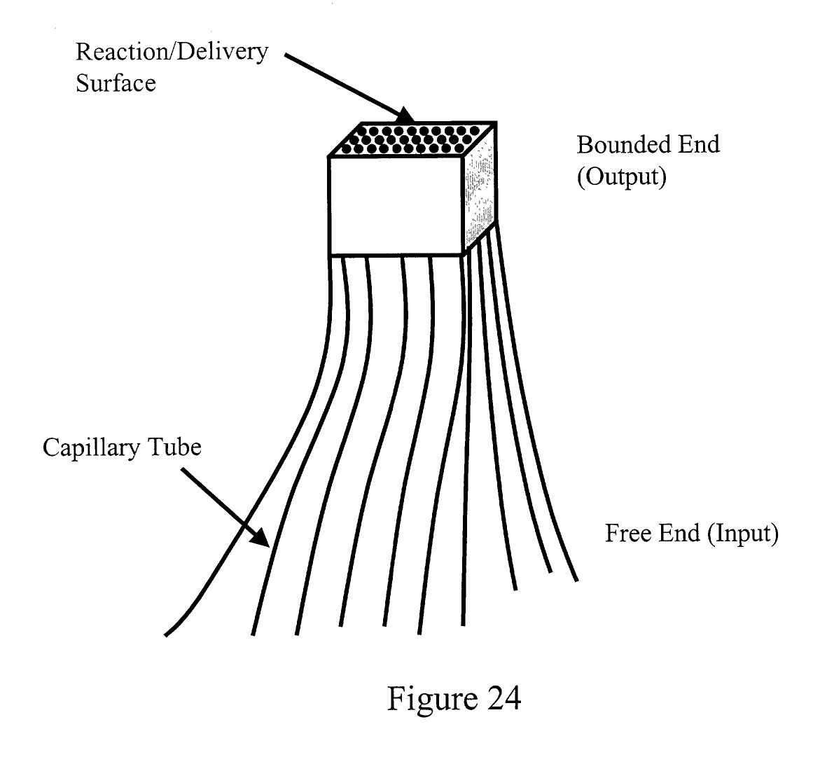

A capillary bundle 110 as depicted in FIG. 24 is fabricated by using capillary tubes, such as those used for capillary electrophoresis. The tubes are bound at one end 102 to form a reaction/delivery head 110. The tubes may be gathered in either a random or an ordered fashion and bound, as discussed in U.S. patent applications discussed above. The minimum number of tubes typically depends upon the number of compounds to be used in a screen. It can be more than 100, preferably more than 10³, more preferably more than 10⁴, more preferably more than 10⁵ or more than 10⁶ or more than 10⁷). The outer diameter of the tubes can range from 5 to 500 micrometers, or preferably 30 to 300 micrometers, or more preferably 40 to 200 micrometers. The inner diameter of the tubes can range from 1 to 400 micrometers, or preferably 5 to 200 micrometers, or more preferably 10 to 100 micrometers. A capillary bundle as described herein may be attached or secured to a frame that is adapted to hold the capillary bundle in a print system. A delivery head may alternatively have a frame that holds a plurality of capillary bundles.

The capillary bundle has two distinguishable ends, the unbound end 204 is referred as the input end, the bound end 202 is referred as the output end. Capillaries on the unbound end 104 may be in contact with a reservoir, such as a microtitre plate well, that holds a chemical compound to be assayed in a way that the capillary can draw fluid from the well. Capillaries on the other end 102 are tightly bound and are typically processed to form a two dimensional array. The minimum number of tubes typically depends upon the number of compounds to be used in a screen (typically 10³-10⁷).

Compound Loading

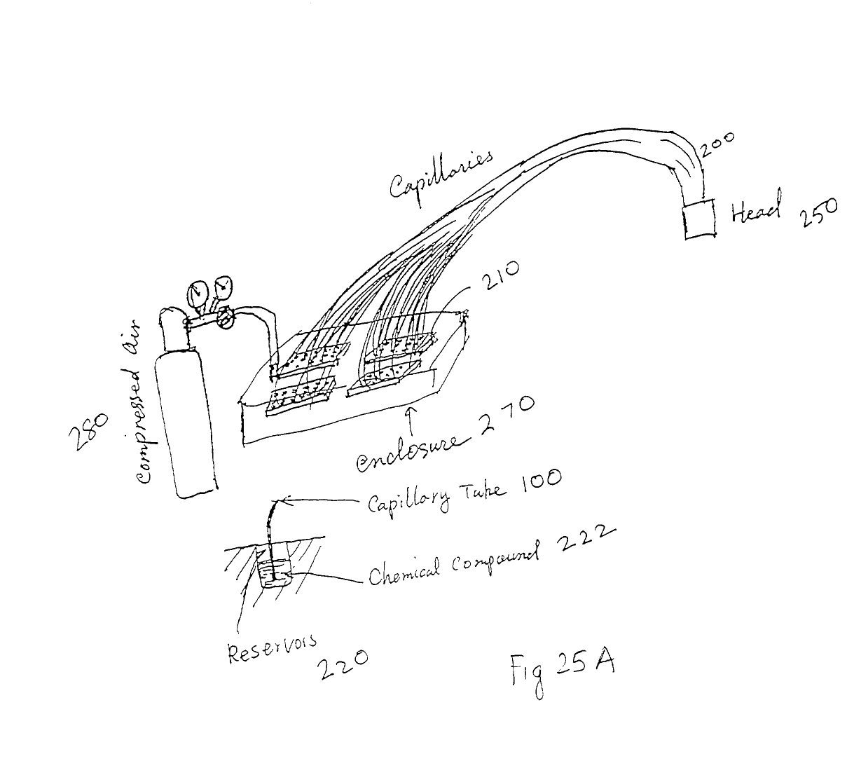

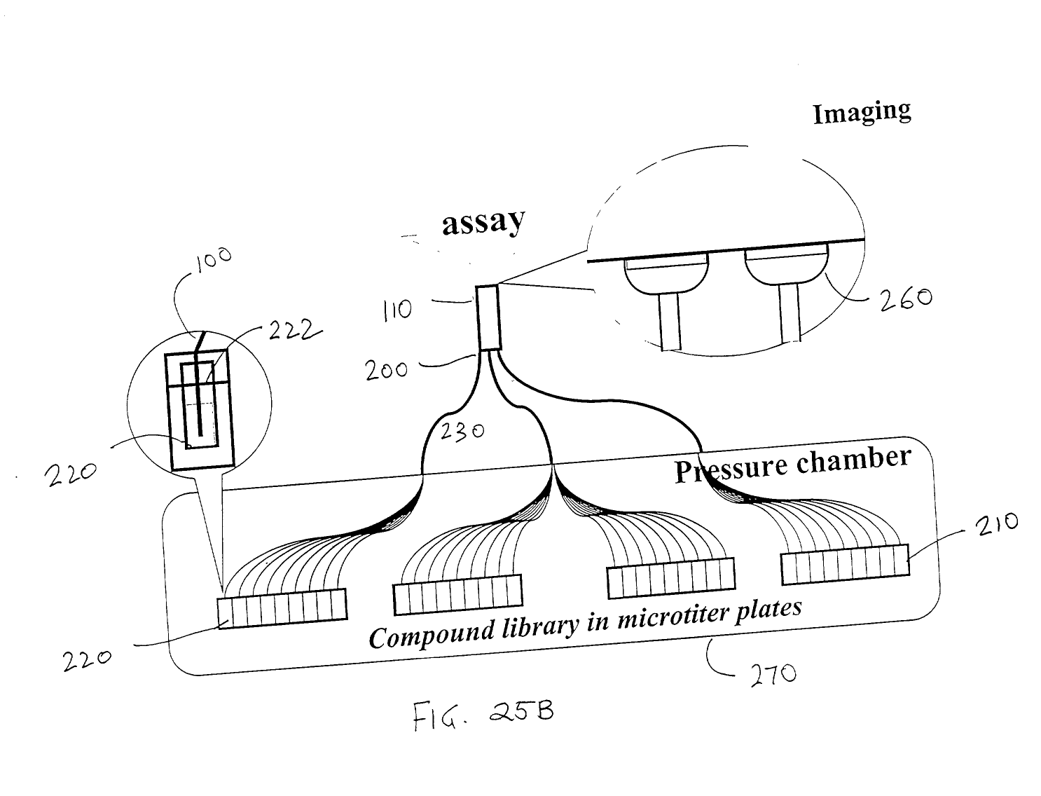

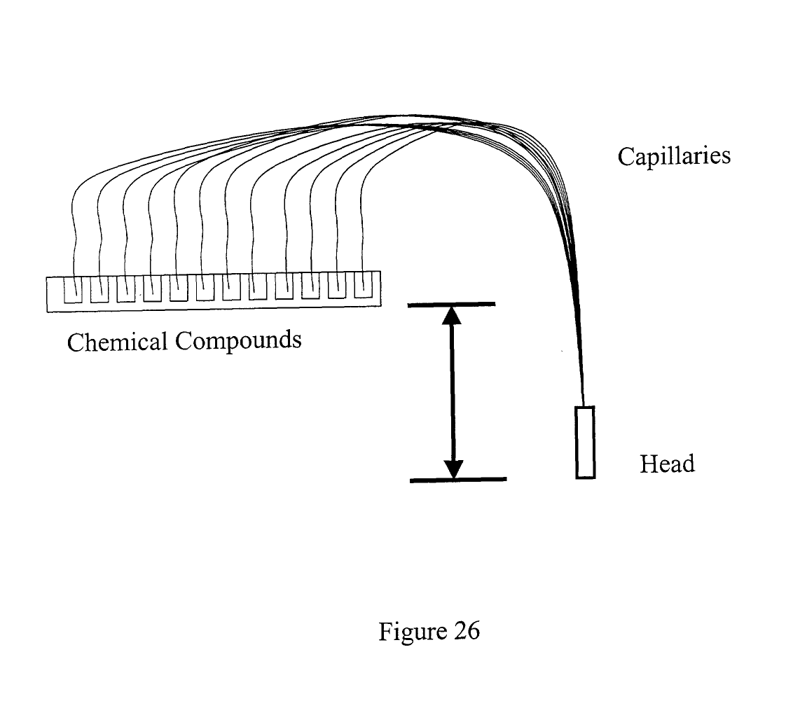

The chemical compounds (including without limitation nucleic acids and their derivatives, lypoproteins, proteins, antigons, antibodies, polysaccharides, lipids, carbohydrates, pharmaceuticals, metabolites, and other organic and inorganic compounds) dispersed in probe fluids are delivered by applying pressure to the reservoirs (as illustrated in FIG. 25A-FIG. 25C) or by gravity (as illustrated in FIG. 26) or by any of the other methods discussed in the pending U.S. and foreign patent applications noted above.

This invention offers several methods to drive fluid from its reservoir into the capillary and towards the reaction chamber. They can be used alone or in any combination of two or more methods in the fluid delivery sub-system. These methods include:

Air pressure: A differential air (or other gas such as nitrogen) pressure can be established and maintained between the proximal and distal ends of the capillary bundles, which will translate into hydraulic pressure to drive the probe fluids.

Gravity: Once the capillaries are filled with the probe fluids, a constant flow can be maintained and controlled by adjusting the vertical positions of the fluid reservoirs, e.g. the microtiter plates, with respect to the position of the reaction chamber.

Electric field: Because fluids are negatively or positively charged, a voltage applied between the reservoir and the reaction chamber can be used to control the flow of the fluid through electrostatic and electro-osmotic force (EOF).

Vacuum: The proximal ends of the capillaries may be placed under relative vacuum. The print head and substrate holder may be placed within a vacuum chamber, and the capillaries may extend through a wall of the vacuum chamber and to the reservoirs. The reaction chamber in this instance preferably extends to the wall of the chamber so that thin capillaries are not exposed directly to vacuum if no liquid flows through them.

Pressure Delivery System

FIGS. 25A-25C illustrate an embodiment of a pressure delivery system. One or more microtiter plates 210 are enclosed in a chamber 270. A chemical compound 222 to be assayed is contained within each reservoir or well 220 of the microtiter plate 210. A free end of a capillary tube 100 connects to the well 220 such that it is in contact with the chemical compound 222 which is preferably dispersed in a fluid form. Multiple such capillaries are bundled 230 at an end 200 distal from the chemical compounds 222 to form delivery head 10. In one embodiment, compressed air or an inert gas such as nitrogen 280 is pumped into a sealed chamber 270 carrying the microtiter plates and a chemical compound 222 from a microtiter plate 220 is translated by hydraulic pressure through the capillary tube to the miniature capillary array. In an alternative configuration, the air pressure at the bundled delivery end 200 is made lower than that at the loose end 104, compound solutions are drawn from the reservoirs to the miniature capillary array.

Gravity Delivery System

In a gravity delivery system illustrated in FIG. 26, the chemical compounds 222 are dispersed in the wells of a microtiter plate 320. Capillaries 310 connect at the free end to the microtiter plate 320 and form a reaction/delivery head 300 at the bound end. By positioning the microtiter plate at a height 340 above the head 300, differential gravitational force is used to siphon the chemical compound from the wells of the microtiter plate 320 to the end of the delivery head 300. The height differential may be transiently operated such that once the compound reaches the end of the reaction/delivery head 300 further flow is ceased by eliminating the height differential. Thus the flow of the chemical compound may be controlled merely by altering the height of the microtiter plate 320 relative to the reaction/delivery head 300.

Electric and/or Magnetic Delivery System

A voltage source may be connected to an electrically-conductive material on a facet of the bundled end 102 and to an electrically conductive material contacting the probe-containing liquid near the loose ends of the capillary tubes 104. A voltage regulator may be used to regulate the voltage and thus the rate of deposition of probe molecules.

Another aspect of the invention may have a bundled end, a plurality of reservoirs, and a magnetic field generator that is positioned sufficiently closely to the bundled end to move a magnetic probe-containing fluid (such as a fluid containing magnetic beads or paramagnetic beads having probes optionally attached to their surfaces) through the capillaries of the bundle.

Design and Fabrication of Delivery Head

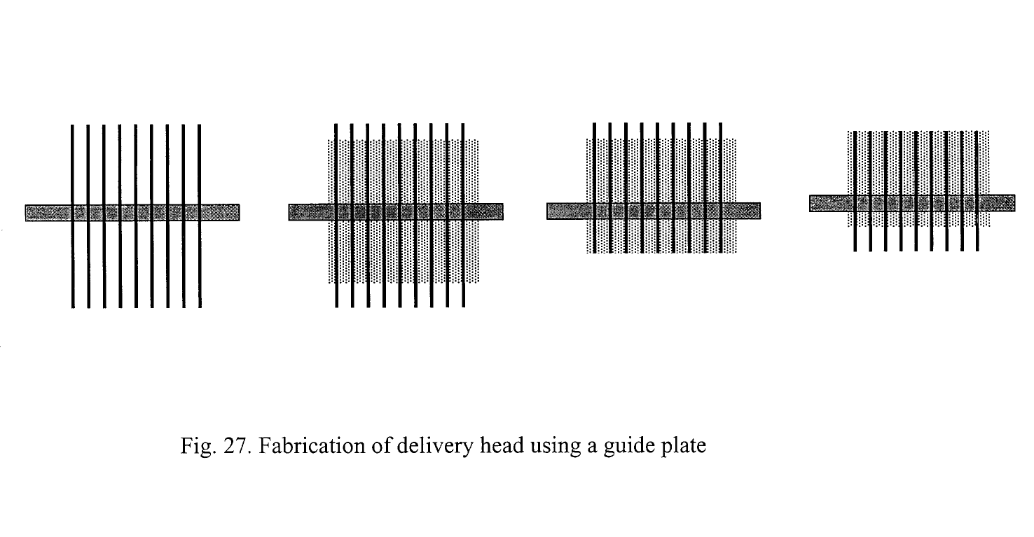

As stated above, the bundled end of the capillary tubes is also termed as delivery head as it directly delivers the compound solution on to the miniature capillary array. This invention provides means at the delivery head to facilitate the delivery. In one embodiment shown in FIG. 27, the delivery head is formed by bonding individual capillary tubes together then cutting and polishing the cut face to form a flat facet. In order to ensure that the compounds are delivered to each assaying site on the capillary array library in parallel without cross-talk, tubes on the facet of the delivery head preferably match the positions of the assaying sites on the capillary array. To ensure this, a guide plate is fabricated which comprises an array of through holes with an exact pattern and pitch as that of the capillary array library. The diameter of the through holes of the guide plate are preferably slightly larger than the outer diameter (OD) of the capillary tubes in the bundle forming the library. FIG. 27 illustrates one embodiment to fabricate the delivery head, where the tubes are first inserted into through holes of the guide plate (FIG. 27a). A bonding material, such as epoxy or ceramic is used to solidify capillary tubes and the guide plate together (FIG. 27b). The solidified bundle is cut at a position very close to the guide plate so that the positions of the tubes are sufficiently close to that of the through holes in the guide plate (FIG. 27c). The end facet is polished and optionally the epoxy or other adherant used to form the solidified mass is etched to form isolated “islands” from the tubes, which prevent fluids in each tube from merging into each other during compound loading (FIG. 27d).

Features of Receiving Capillary Array Compound Library

Fluidic features are built into the capillary array compound library that receives and stores the compound fluids. These features will be presented briefly here and in detail in the next section.

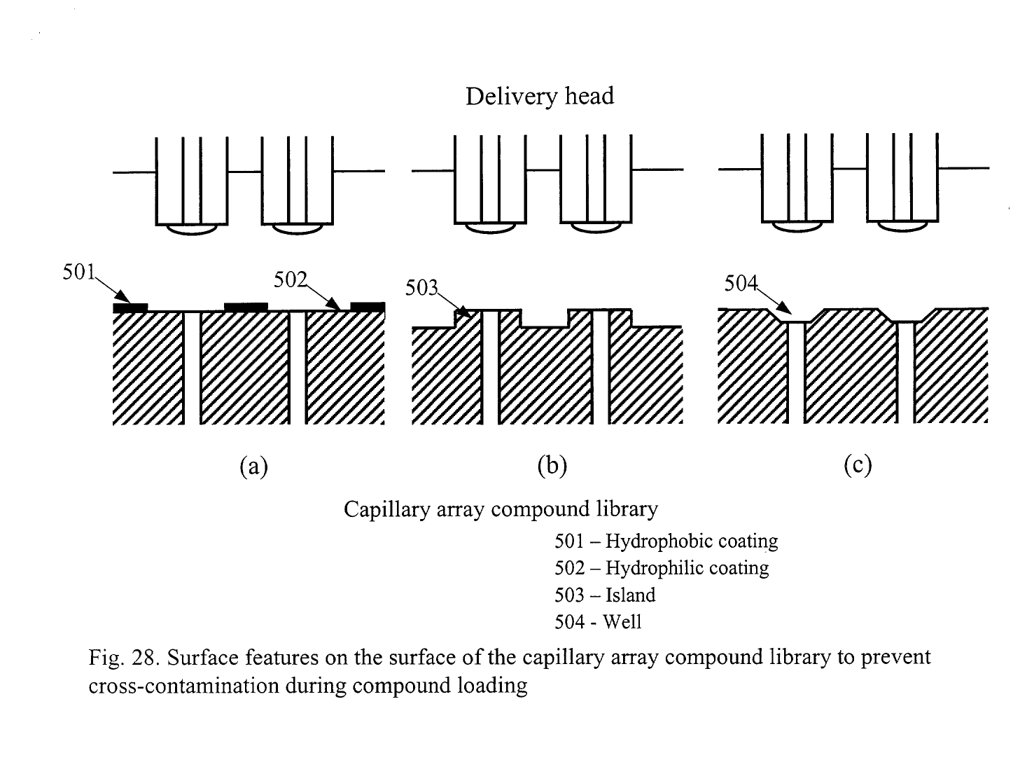

The capillary array, compound library comprises a substrate having a large number of assaying sites that terminate at a common surface, which is termed the “assay surface”. The assaying sites may be through holes that pass through the substrate and may have the same cross-sectional area from one end of the substrate to the other. Alternatively, the assaying sites may be reaction chambers that have a larger cross-sectional area than the through holes mentioned above. Each assaying site comprises at least one chamber capable of storing compound solutions, which may be the through hole discussed above or a portion of the through hole. The compound loading station deposits the compound solutions on the assay surface and the solutions are drawn into different compound storage chambers by capillary force or pressure. Fluidic features may be formed on the assay surface to isolate deposited solutions so that they will not merge into each other causing cross contamination or fluid “cross-talk”. A number of embodiments of these features are illustrated in FIG. 28, which include hydrophilic patches (FIG. 28a) or geometric structures, such as, islands (FIG. 28b) or wells (FIG. 28c), that optionally mate with the delivering capillaries from the loading station.

(c) Desktop HTS Station

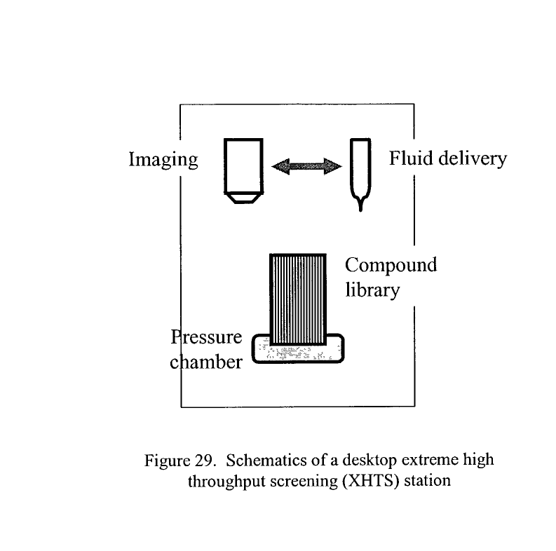

Once the user receives the miniature compound library held in a capillary array, he/she can insert it into the desktop HTS station, as shown in FIG. 29. One end of the array is pneumatically or hydraulically connected to a small precision pressure chamber, the other end (or termed “assay end”), where the micro-reaction wells are located, is accessible to liquid handling and imaging arms.

During screening operation, the target reagents, such as enzymes and substrates are universally applied to the micro-reaction wells at the assay end of the capillary array by a fluid delivery nozzle or nozzles. Compound solutions can be pumped to the wells from the through holes by applying a suitable pressure differential between the pressure chamber and the assay end. Alternatively, a suitable inert liquid that is immiscible to the compound solutions can be filled in the pressure chamber. Solutions in the through holes can be pumped out or back by displacing the volume of the fluid in the pressure chamber. In both cases, mixing and reaction occur in the micro-reaction wells and can be detected there using imaging equipment.

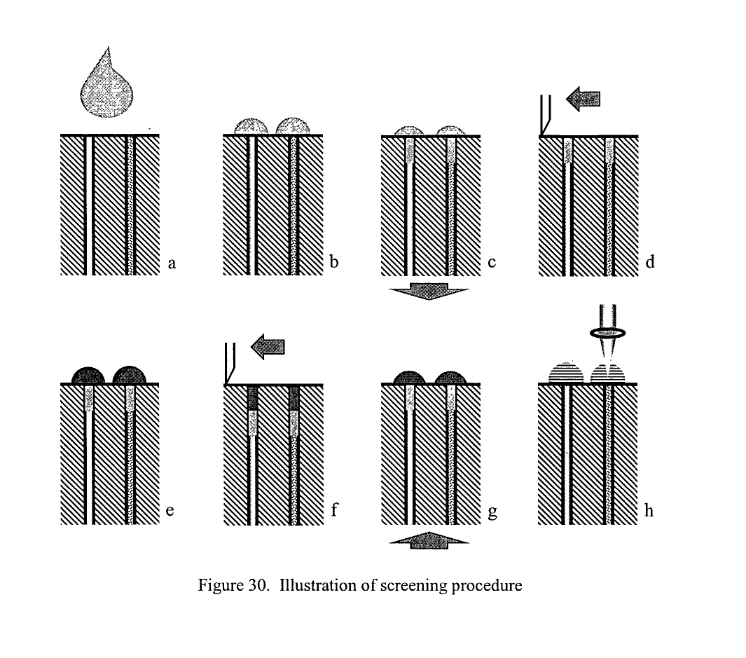

FIG. 30 illustrates a typical sequence of operations during an enzymatic assay screening using the virtual well configuration illustrated in FIG. 4. Firstly, the substrate is applied to the assay end in a flooding manner (FIG. 30a). Droplets of substrates are retained in the wells after excess fluid runs off (FIG. 30b). Second, a negative pressure is applied to the pressure chamber to draw a controlled amount of substrate into the through hole (FIG. 30c). Third, the excess substrate fluid at the facet surface is removed by various means including wiping, tissue absorption, blowing or a vacuum force, which is smaller than the retaining capillary force in the through hole (FIG. 30d). Fourth, enzyme is applied to the assay end in a way similar to how the substrate is applied (FIG. 30e). Fifth, a defined amount of enzyme is sucked into the through holes and the excess enzyme on the facet is removed in the same way as the excess substrate described above (FIG. 30f). Sixth, defined amounts of enzyme, substrate and compound are pushed out of the through hole into the micro-reaction well by applying a controlled positive pressure in the pressure chamber (FIG. 30g). The fluids mix and incubate in the well. The result of the reaction can be detected using optical methods (FIG. 30h), which may include laser scanning based technology similar to that used in microarray readers. The reading can be conducted above the library array at the assay side. It can also be done from the other side if the array substrate material is transparent or if the fiber optic capillaries described in previous U.S. patent applications Ser. Nos. 09/791,994 and 09/791,998 are used to construct the array. After imaging, the assay end of the array can be washed, and excess fluid removed. The array is now ready for the screening of the next target.

The steps of substrate and enzyme application may be switched depending on the specific assay design. Alternatively, enzyme and substrate may be mixed before application to the reaction wells.

Because the micro-reaction well is always linked to the through hole through which additional buffer fluid can be supplied, the evaporation in the micro-reaction well can be well compensated. Cellular based assays can be conducted in a similar way using relatively larger reaction wells.

When the real well configuration illustrated in FIG. 3 is used, the operational steps are very similar to the procedure above except the excess fluid in the micro-reaction well typically is not removed by wiping. The other methods including blowing, tissue absorption and vacuum sucking may be better suited.

As illustrated in FIG. 2c, different solutions may be stored in the same through hole in the capillary array. This particular format may have a number of different applications. In some complex screen assays, reaction between the target reagent and different chemical compound may require different conditions, such as different pH values, and these conditions may have to be set up at different times during the incubation process. In this situation, one or multiple conditioning fluids may be loaded in different sections along the same through hole behind the compound. These solutions can be injected into the micro-reaction well during different stages of incubation. This will provide much more flexibility in the assay development and is especially useful in protein array applications where each protein-protein interaction may require different fluid conditions.

In a different application of the above format, different chemical compounds may be loaded in the same through hole. They may be separated by only a gas bubble or gas bubbles plus a section of cleaning agent. The function of the cleaning agent is to remove any residuals of the first compound from the inner wall of the hole so that the subsequent compounds will not be contaminated. This application allows great expansion of the storage capacity of the capillary bundle. The appearance of the bubble in the micro-reaction well can easily be detected using conventional machine vision systems and can be used by the system to automate the process.

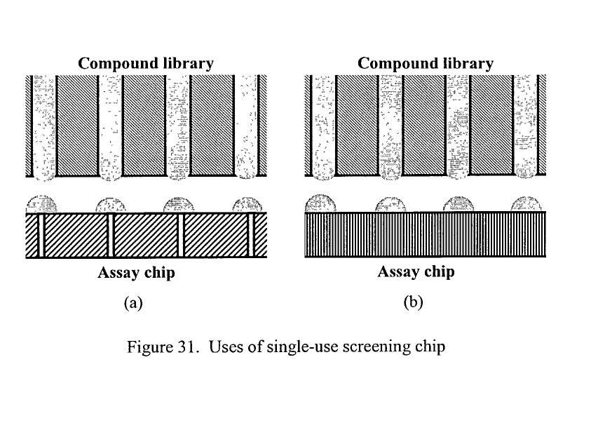

In a different screening method, the assay is not conducted directly on the facet of the capillary array holding compound library, but on a separate “assay chip” instead. The “assay chip” has its own through hole array. The pitch and pattern of the through hole on the assay chip may be exactly the same as that of the compound library, as shown in FIG. 31a, or they can be much denser than that of the library. Micro-reaction wells are fabricated on one of the facet of the assay chip (FIG. 31b). FIG. 31 shows that the compound solutions are loaded to the assay chip from the micro-reaction well side. They can also be loaded from the other side of the assay chip. The solutions are sucked into the through holes due to capillary force.

One facet of the assay chip is pneumatically connected to a precision pressure chamber while the other is available for fluid application and imaging. The typical steps of screen assay using assay chip is very similar to that using compound library directly. The only difference is that the compounds are now delivered to the reaction wells by aligning the through holes of the compound library to the reaction wells on the assay chip. The assay chip may be formed by forming a capillary bundle as described above and then cutting the bundle to form a chip of desired thickness (based on the volume of the holes in which reactions are to occur). The chip so formed has a pattern of holes that exactly matches the pattern of capillaries of the bundle.

This invention provides a desktop-sized screening station that performs fully automated HTS operation in a personal setting, which includes the following basic functionalities: the station loads the capillary array compound library in one or multiple cartridges if supplied on multiple chips, and the station stores the cartridge in a suitable, controlled environment chamber.

The station accepts and routes additional reagents needed for the HTS assay, which usually include an enzyme, a substrate and buffer, and the station may pre-process these reagents, which may involve dilution and pre-mixing.

In cases where the compound is dried in the through hole of the library before shipping to users, the screening station may redissolve the compound in pure DMSO. Preferably, the concentration of DMSO or other polar solvent is no more than 1%.

The station delivers reagents to the capillary array compound library, facilitates reagent metering by removing excess fluids by e.g. tilting the library, vacuuming, squeegeeing, and/or air-blowing the surface. The station may also initiate mixing of these reagents with compounds in separated mixing/reaction micro-chambers of e.g. a capillary array.

The station provides suitable environmental chambers for the capillary array to incubate. The station has an integrated detection system to detect signals indicating the results of the assay. The station has the capability to clean and regenerate various surfaces that have been used by previous screening assays and prepare for the next HTS operation.

Pre-Screen Environmental Chamber

The shipping package seal will be opened before the capillary array cartridge is inserted in the pre-screen environmental chamber. The chamber provides a clean, DMSO rich and cooled, preferably to 4° C., environment to ensure that the compound solutions stored in the through holes of the capillary array remains fully effective over a prolonged period of time before screening.

Reagent Cartridge

In one particular embodiment of the screening station design, a single-use reagent cartridge is provided, which has separate reservoirs for multiple reagents needed for the HTS assay. Reagents can be loaded into the cartridge outside the machine and then the cartridge may be inserted into a designated port on the screening station. Pre-dilution or mixing of reagents, if needed for the assay can also be conducted on the cartridge. This can reduce the burden of cleaning after the HTS assay.

Re-Dissolution of Dried Compounds

This additional step is only needed if the compound is shipped dry in the library. Pure DMSO may be introduced to the capillary compound library, which is drawn into the compound storage chamber by capillary force. Excess DMSO is removed. After a certain incubation period, the compound powder will be re-dissolved into the DMSO solution and ready for HTS assay.

Assay Station

The invented screening station may provide a mechanism to remove individual capillary arrays from a cartridge without the need for manual handling. The cartridge is loaded on an assaying station, which has fluid handling capabilities to enable the delivery of multiple reagents from their storage cartridge described above to the capillary array, removal of excess fluids after reagent metering and mixing them with compounds in different mixing chambers. This invention provides a number of different fluid handling mechanisms, which are related to the structure of the through holes in the capillary array or library.

Because the reagents used for HTS assays are common to every compound holding through holes in the capillary array, one method to deliver the reagent to the capillary array is to flood the reagent liquid onto the assay surface of the capillary array. The fluid metering devices built on the capillary array, such as the virtual or physical wells described previously, will hold a designated volume of fluid and the excess fluid will be removed by e.g. tilting the substrate to allow excess fluid to run off.

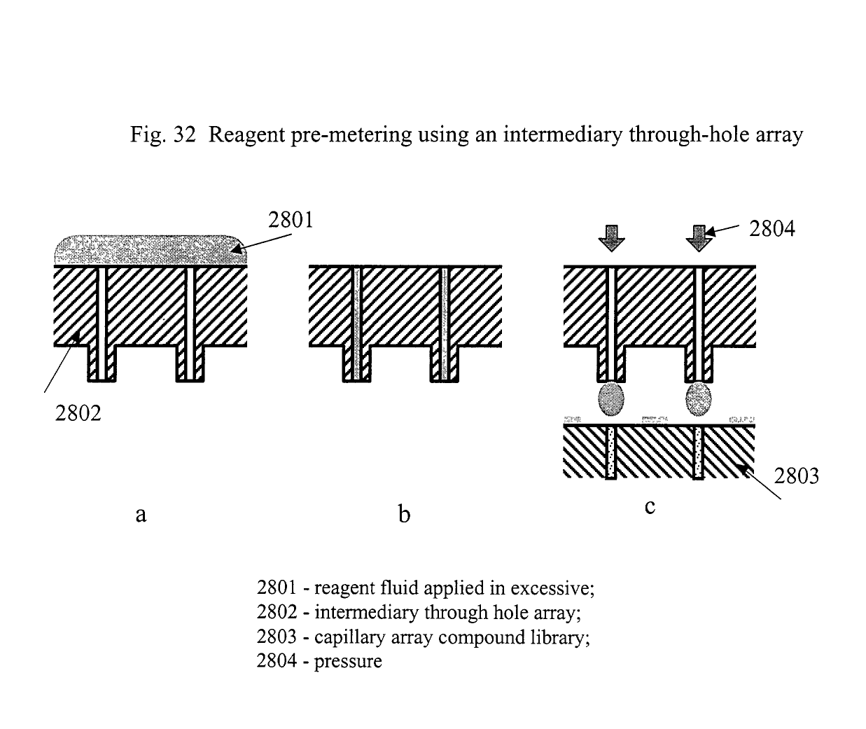

Another delivery method is a two-stage approach. As illustrated in FIG. 32, a chip having an array of through holes serves as an intermediary liquid delivery device. The through holes in the chip spatially match the compound holding sites in the capillary array compound library. The inner volume of each through hole is slightly larger than the reagent volume needed for mixing with each compound. The function of this through hole chip is to pre-meter and distribute the reagent to each compound contained in the chip or library. The bulk reagent is delivered to the top surface of the chip in a flooding fashion. The reagent solutions fill each through hole by capillary force (FIG. 32a). The excess reagent fluid is then removed from the top surface of the chip as described previously (FIG. 32b). Further, the through holes in the chip are aligned with the compound holding through holes in the capillary array or library. The reagent is driven out of the intermediary chip onto the capillary array compound library by pressure (FIG. 32c). Because the reagent is pre-metered for each compound, the amount of excess fluid is greatly reduced, which reduces the chance of cross-contamination between compounds.

The following are a number of examples describing detailed steps of typical HTS assays carried out in a screening station. The enzymatic assay involves adding an enzyme and a substrate in two steps and mixing them with the compound contained in the capillary. Other assays can be conducted in a similar fashion. Most of these assay steps have been described in previous sections. the following explanation provides a more integrated presentation of the operation of the entire system.

Enzymatic Assay with Multiple Use Compound Library

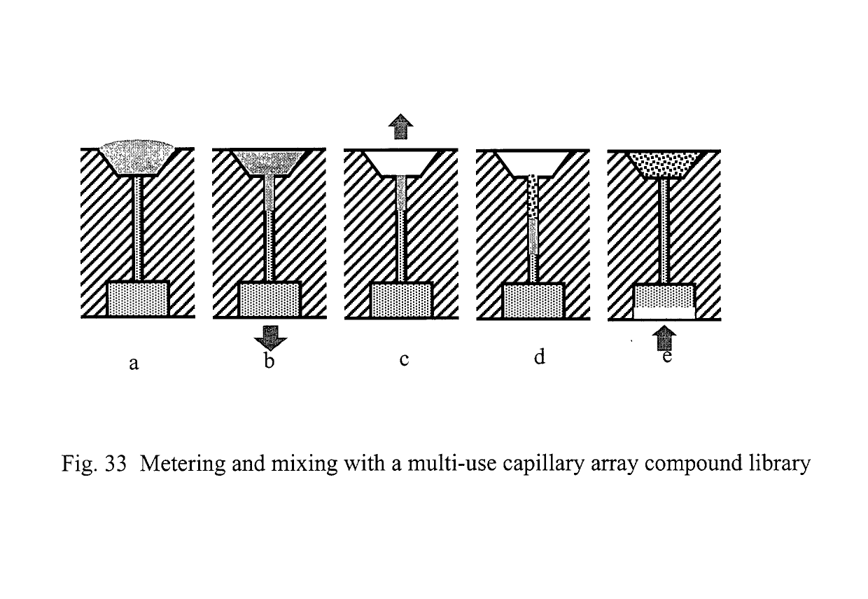

FIG. 33 illustrates the operational steps to carry out an enzymatic assay using a capillary array compound library designed for multiple uses. The through hole structure comprises a micro-reaction well linked to a large compound reservoir through a long and narrow path. First, the enzyme solution is deposited on the assay surface in bulk, filling the micro-reaction wells (a). Second, a negative pressure is applied to the reservoir side to draw a defined amount of enzyme into the narrow path region to dispense some of this compound(b). Third, the excess enzyme in the well is removed by vacuum aspiration from the top (c). The same operations from 1^{st} to 3^{rd} step are carried out for substrate solution in 4^{th} to 6^{th} steps. As a result, there are two short slugs of enzyme and substrate fluids in the narrow path as well as some assaying compound (d). Seventh, a positive pressure is applied to the reservoir side which pushes both fluids plus a defined amount of compound out into the micro-reaction well where they mix, incubate and are read by the detection system. After readout, the mixture in the micro well is removed and washed with buffer. The device is ready for the next screen. In this particular capillary array compound library, volume metering is achieved through precise pressure acting on the narrow path or channel, which functions as a fluid regulator.

Enzymatic Assay with Single Use Library and Virtual Well Metering

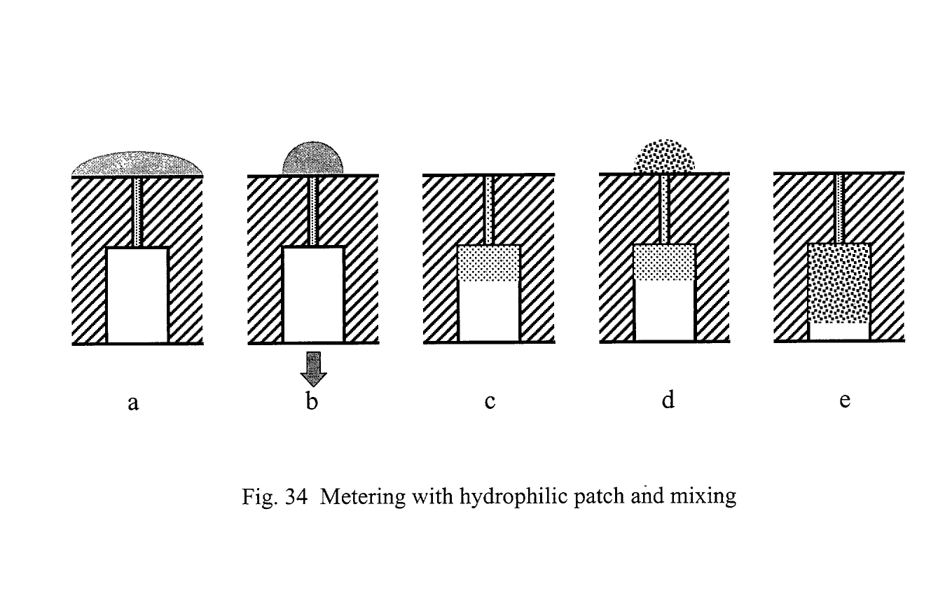

FIG. 34 illustrates the operational steps to carry out an enzymatic assay using a single use capillary array compound library. The through hole structure comprises a “virtual well” on the assay surface. The mixing/reaction chamber is linked to the virtual well through a capillary portion, which stores the compound. First, the enzyme solution is deposited on the assay surface in bulk (a). Second, the surface is tilted to remove the excess fluid. A defined volume droplet is retained by the hydrophilic patch around the through hole entrance (b). Third, a negative pressure is applied to the mixing chamber side to draw in the entire droplet through the compound chamber into the mixing chamber. The enzyme will start mixing with the substrate (c). Steps 4^{th} to 6^{th} repeat steps 1¹ to 3^{rd} (omitting the analogous step to step (a) from the figures) but use the substrate solution in place of the enzyme solution. All three reagents mix in the mixing chamber (d).

Enzymatic Assay with Chamber Metering

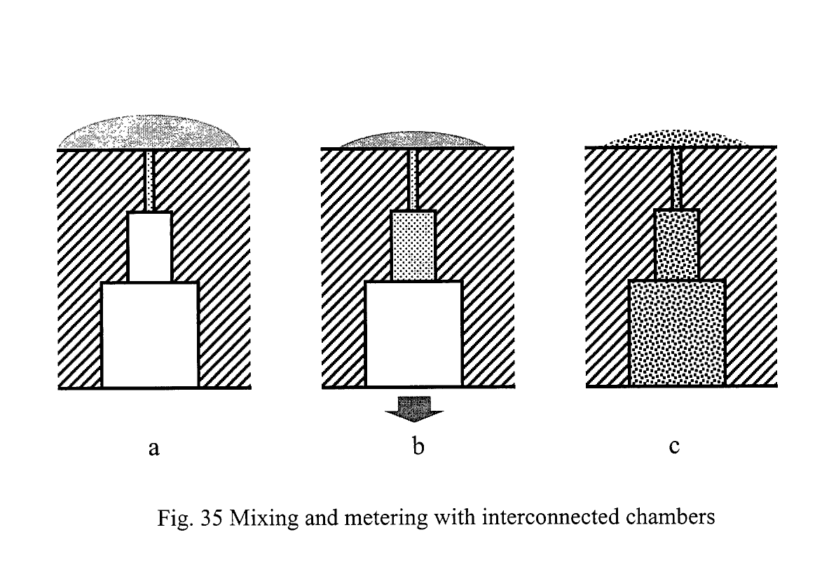

FIG. 35 illustrates the operational steps to carry out an enzymatic assay using a single use capillary array compound library. The through hole structure comprises three interconnected chambers. The thin capillary chamber closest to the assay surface is used to store the compound. First, the enzyme solution is delivered to the assay surface in bulk (a). Second, a short duration of negative pressure is applied to the mixing chamber side, which breaks the fluid barrier formed by the large and abrupt expansion between the compound chamber and its adjacent enzyme mixing chamber. The fluid fills the second chamber due to capillary force drawing in a define the volume of enzyme, which mixes with the compound in the enzyme mixing chamber (b). After removing excess enzyme from the assay surface, steps 3^{rd} and 4^{th} will be carried out to the substrate similar to steps 1^{st} and 2^{nd}. The fluid barrier between the enzyme mixing chamber and final mixing chamber is overcome, and the compound, enzyme and substrate mix in the two chambers (c).

Enzymatic Assay Using Through Hole Metering

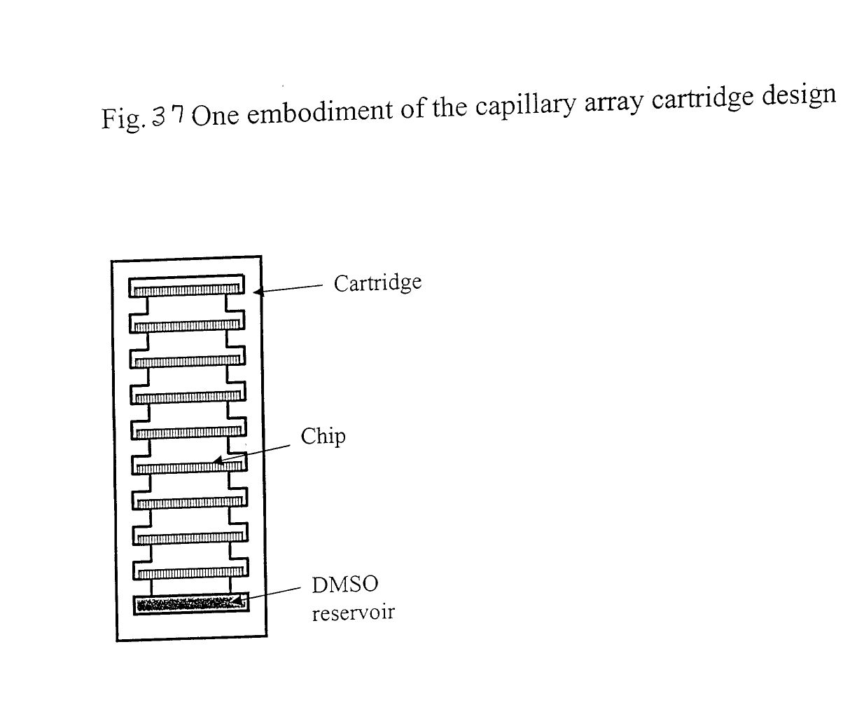

FIG. 36 illustrates the operational steps to carry out an enzymatic assay using a single use capillary array compound library chip and multiple separate reagent metering chips. The through hole in the library chip comprises two interconnected chambers. The thin capillary chamber closest to the assay surface is used to store the compound. The much larger chamber is used for reagent mixing and reaction. Separate enzyme and substrate metering chip are constructed which have a through hole array at the same pitch and spatial pattern as the through holes in the library chip. The inner space of each through hole in the enzyme or substrate metering chip is designed to be same as the volumes of enzyme and substrate solutions required for the assay, respectively. In most HTS applications, the volumes of enzyme and substrate 50 to 500 times larger than that of the compound. Therefore, it is desirable that the diameter and volume of the through holes in the enzyme or substrate metering plate is much larger than that of the compound storage chamber in the library chip. As illustrated in FIG. 37, the enzyme and substrate solutions are first delivered to through hole plate A and B, respectively and metered in a process described previously. Then the through hole in plate A is aligned with a compound storage chamber on the library chip and a fluid connection is established (a). Second, a negative pressure is applied to the mixing chamber side to draw not only all the compound but also all the enzyme in the through hole a separate chip into the mixing chamber (b). Then Plate B is aligned to the library chip (c) and a negative pressure at the mixing chamber side (or a positive pressure at the Plate B side) is used to draw (or push) all substrate into the mixing chamber (d), where the three solutions will mix and incubate.

Heterogeneous Protein or Cell Assays

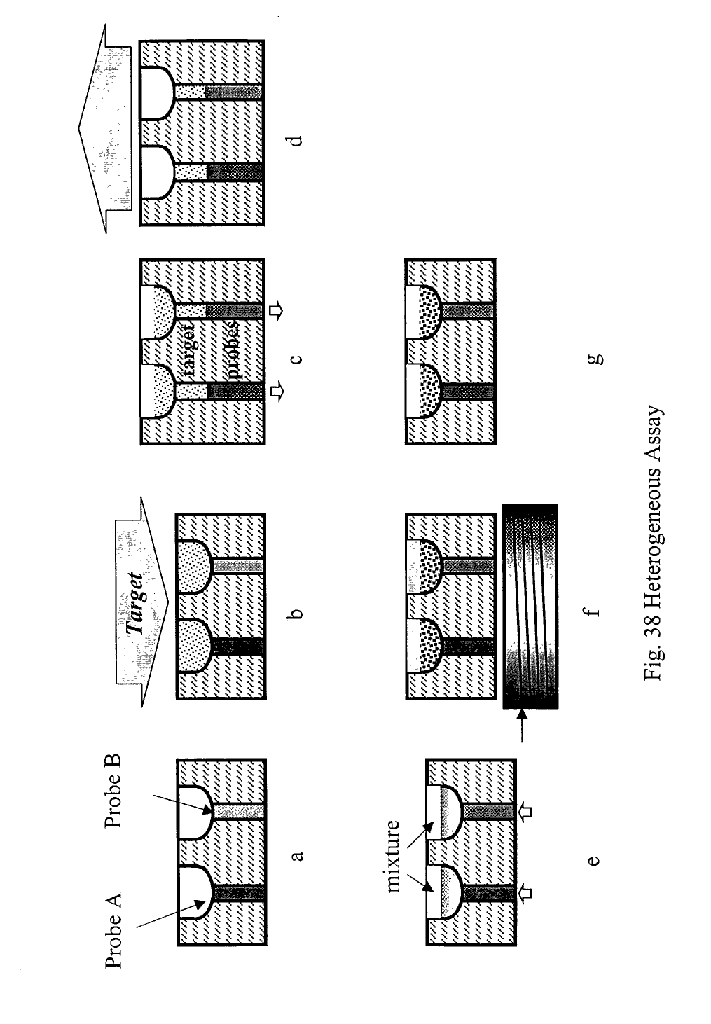

The invention can readily be applied in protein array fabrication, assaying and readout system. FIG. 38 illustrates an embodiment of an assay involving protein arrays or cell arrays. A library of antigens or antibodies is attached to magnetic beads 460 (Dynal Corporation) using standard biochemical protocols. The method discussed above is used to mix the sample and proteins or cells of the library. The reaction head may be sealed using e.g. a glass or polymeric plate 470 as illustrated at step (e), and the reaction head may be transported to a separate magnetic head 480, where the plate is removed, a washing fluid is placed into the chambers as part of the washing cycle, the beads are subjected to a magnetic field generated by the head (e.g. an electromagnet), and the fluid is removed by aspirating it but the beads are held in place by the magnetic field. Washing steps are necessary in heterogeneous assays, and washing is greatly facilitated by use of paramagnetic beads that are retained in the reaction chamber by the magnetic field generated by the electromagnet when the wash liquid is removed. The system is then demagnetized, and the reaction head is moved to a position for imaging e.g., using a fluorescence scanner. Once scanning is completed, the magnetic beads are aspirated from the reaction chambers, the reaction chambers are washed as described previously, and the reaction head is prepared for another cycle.

Incubation Chamber

After mixing, one or multiple capillary array compound libraries can be placed in an incubator, which maintains a high humidity and suitable temperature for a designated duration for reaction incubation.

Detection System

The screening station provided by the invention provides an integrated detection system to detect optical signals generated by the HTS assay. Detection of biomolecular reactions on the invented system may be carried out using colorimetric, fluorometric, electrochemical, and/or electronic detection labels. Optical detection modes may include absorption, calorimetric, chemical luminescence, fluorescence intensity, FRET, time-resolved fluorescence and fluorescence polarization. When the reaction occurs in the reaction well or the virtual well on the substrate surface (using surface tension to restrict fluid flow), the reaction may be followed using standard detection techniques such as those involving optical, CCD, CMOS or laser optics. Where the reaction occurs within the capillaries and the reaction product is not extruded from the through hole (or the reaction is followed in real time) a variety of methods may be used to extract the signal from within the capillary. Use of an optical fiber capillary coupled to a detection (CCD, C-MOS) device at a remote end will allow a technician to follow a reaction. Alternately, the walls of the capillary may be lined with light reflective material (as shown in FIG. 20) to amplify a light signal such as that generated by a fluorescent probe. In another embodiment, the substrate itself may be fabricated from a transparent material. Examples of some detection labels suitable for the present invention are discussed below:

Fluorescent Probes

Interaction of the target and chemical compounds can be assayed by detecting the fluorescence emission (intrinsic or extrinsic probes) of a target system labeled with fluorescent molecules such as, e.g., DAPI, Texas red and fluorescein. The detection system can be a charged coupled device (CCD) based fluorescence imaging system. In one illustrative but non-limiting example of CCD-based fluorescence imaging and analysis, fluorescence images of 5 mm×7 mm regions of the reaction heads or through hole plates are obtained using a 1× magnification imaging system coupled to a 12 bit CCD camera (e.g., Photometrics KAF 1400 chip). Excitation light, supplied from a mercury arc lamp equipped with a computer controlled filter wheel, is projected onto the reaction head using a quartz prism. After impacting the reaction head the light is reflected to the CCD detector. A multiband pass filter (e.g., P8100, Chroma Technology, Brattleboro Vt.) is used in the emission light path. Exposure times are less than one second for DAPI, and between 0.5 and 2 sec for fluorescein and Texas red. Images are analyzed with software that segments the array targets based on the DAPI image, subtracts local background, and calculates several characteristics of the signals for each target including the total intensity of each fluorochrome, the fluorescein/Texas red intensity ratio, and the slope of the scatter plot of the fluorescein and Texas red intensities for each pixel.

A microarray or compound library comprising a random bundle may have software associated with it that provides data which correlates the identity of the target or probe molecules with a particular location on the reaction head, as discussed above. The software may be provided as a database providing this correlation and may be on a portable medium such as a CDROM or may be downloaded to a user's equipment via a telephone line, cable modem, satellite link, or other form of data communication. The software may also be programmed into an EPROM located on the library. The software may be loaded into a computer or into dedicated equipment associated with a scanner, such that the hybridization pattern read by the scanner can be translated into information on the target molecules or probe molecules that have hybridized (or otherwise associated) on the substrate.

Fluorescence Quenching and Light-up Probes

In the systems of the present invention, the analyte-probe moiety is detected. There are three basic methods of detection: first, no label, in which an intrinsic property of the probe-analyte structure which is different from that of probe or analyte alone is detected; second, a single label, either on probe or analyte, either produces a signal which may be measured after unbound label is removed, or an existing signal is altered in a measurable way upon formation of the probe-analyte structure, thus obviating the requirement of removal of unbound label; third, label pairs, in which at least one label on the probe and one label on the analyte interact upon binding to produce a signal, which also obviates the need for removal of unbound label. Any of these may be used in the methods of the invention.

Label One Member of a Pair

Several methods have been developed and are known to those of skill in the art for using a single label which is altered upon formation of the analyte-probe pair. For nucleic acids, the use of light-up probes in nucleic acid analysis allows one member of a probe-analyte pair to be labeled in such a way that binding of probe and analyte results in a large increase in fluorescence signal. The use of such probes is known in the art and discussed in, e.g., U.S. Pat. No. 6,329,144; Svanvik et al., Anal Biochem 281:26-35 (2000). Other methods include probes composed of an oligodeoxyribonucleotide equipped with a ruthenium complex, where hybridization can be demonstrated from measurements of the probe fluorescence lifetime (Bannwarth et al., Helvetica Chimica Acta, 71, 2085, 1988); a probe composed of a DNA-chain modified with a metal-ligand complex whose fluorescence intensity increases upon hybridization (U.S. Pat. No. 5,157,032); a probe composed of an oligonucleotide modified with pyrene, which under optimal conditions gives a 20-fold increase in fluorescence upon hybridization (Yamana et al., Nucl. & Nucl. 11 (2-4), 383, (1992); probes composed of an oligonucleotide and an asymmetric cyanine dye, whose fluorescence properties, such as fluorescence polarization, fluorescence lifetime and fluorescence intensity, are changed upon hybridization (EP 0710 668A2, U.S. Pat. No. 5,597,696; Ishiguro et al., Nucl. Acids Res. 24, 4992, (1996). Methods in which two probes are used to analyze a single analyte also are applicable, such as a probe based on simultaneous hybridization of two DNA-based probes to close-lying sequences, where one probe is modified in the 3′-terminus of the DNA chain with a donor fluorophore and the other probe is modified in the 5′-terminus with an acceptor fluorophore. When they are in proximity fluorescence energy is transferred from the donor to the acceptor fluorophore, which can be detected. The fluorophores are far apart in solution, but are brought together when the probes hybridize to TS by binding with the 3′-terminus of one probe next to the 5′-terminus of the other probe. See, e.g. Heller et al., (EPA 070685) and Cardullo et al., (Proc. Natl. Acad. Sci. USA, 85, 8790-8794, 1988).

Interacting Label Pairs

In one mode of detection, the probe and the analyte each comprises a member of an interacting label pair. The members interact when in close proximity, such that association of the members on the two probes results in generation of a signal. By “signal” is meant a measurable characteristic. The signal may increase or decrease upon association of the members of the interacting label pair. For example, if the interacting label pair comprises a fluorophore and a quencher, association of the members of the pair generates a detectable signal due to a decrease in light energy emitted by the fluorophore in response to illumination. Or, for example, if the interacting label pair comprises subunits of an enzyme, association of the members of the pair generates a detectable signal which is an increase in the rate of the reaction catalyzed by the enzyme. Each member of the interacting pair may comprise one or more than one molecule or structure. The change in signal may be all-or-none (for example, if the moieties are an enzyme-inhibitor pair, where the enzyme is either active or inactive) or vary over a range (for example, if the moieties are a fluorophore-quencher pair). The change is characteristic for the moieties (labels) employed. In some embodiments, two or more kinds of interacting label pairs may be used in a single sample in order to differentiate, e.g., different target analyte acid sequences. The detectable signal may be, e.g., a characteristic light signal that results from stimulating at least one member of a fluorescence resonance energy transfer (FRET) pair. Another example of a detectable signal is a color change that results from the action of an enzyme/suppressor pair or an enzyme/cofactor pair on a substrate to form a detectable product. In some embodiments, the signal is a reduction or absence in detectable signal.

Various combinations of moieties (labels) which are capable of producing a detectable signal which differs depending on their degree of proximity, can be used. Any combination or number of moieties (labels) which interact so as to produce a measurable change upon change in the proximity of the moieties (labels) is sufficient; hence, more than one pair of moieties (labels) may be used. Nor is it required that there be a one-to-one correspondence between members of an interacting label pair, especially where one member can affect, or be affected by, more than one molecule of the other member.

Interacting label pairs useful in the present invention are known in the art, see, e.g., U.S. Pat. Nos. 5,688,648 (Mathies et. al) ; 5,340,716; 3,999,345; 4,174,384; and 4,261,968 (Ullman et al.); 4,996,143 and 5,565,322 (Heller et al.); 5,709,994 (Pease et al.); and 5,925,517 (Tyagi et al.). Examples of suitable moieties (labels), in which one member quenches another, include a fluorescent label, a radioluminescent label, a chemiluminescent label, a bioluminescent label, an electrochemiluminescent label, and an enzyme-inhibitor combination. In some embodiments, the interacting moieties (labels) may generate little or no signal when in close proximity and generate a greater signal when separated. In other embodiments, the interacting moieties (labels) produce little or no signal when separated, and a greater signal when in close proximity. Examples of the latter such moieties (labels) are an enzyme and its cofactor and fragments or subunits of enzymes that must be close to each other for the enzyme to be active.

If fluorescent labels are used, labels are chosen such that fluorescence resonance energy transfer is the mode of interaction between the two labels. In such cases, the signal generated by the association of the labels could be an increase in the lifetime of the excited state of one label, a complete or partial quenching of the fluorescence of one label, an enhancement of the fluorescence of one label or a depolarization of the fluorescence of one label. The labels could be excited with a narrow wavelength band of radiation or a wide wavelength band of radiation. Similarly, the emitted radiation could be monitored in a narrow or a wide range of wavelengths. Examples of such pairs are fluorescein/sulforhodamine 101, fluorescein/pyrenebutanoate, fluorescein/fluorescein, acridine/fluorescein, acridine/sulforhodamine 101, fluorescein/ethenoadenosine, fluorescein/eosin, fluorescein/erythrosin and anthranilamide-3-nitrotyrosine/fluorescein. Other such label pairs will be apparent to those skilled in the art.

Various combinations of dye moieties (labels), which are capable of energy transfer when in close spatial proximity, can also be used. For example, interacting moieties (labels) may be a donor-acceptor dye pair, capable of energy transfer when in close spatial proximity. Label 1 may be a fluorescent dye and label 2 a quencher which is able to absorb the fluorescence signal of label 1 by an energy transfer mechanism. Alternatively, the moieties (labels) may be ligands for reporter molecules which can interact with each other when brought in close spatial proximity, the interaction of which prevents or enables activity of one of the reporter molecules. Examples for suitable combinations of reporter groups useful for the methods of the invention are enzyme-inhibitor combination, reporter molecules which when reacting with one another form an active enzyme molecule, and the like. The association of the two interacting reporter groups is detectable and indicative of the presence of one or more target analyte(s) in a sample, the quantity of analyte(s) in a sample or degree of identity of the analyte with a reference, e.g. the degree of identity of a sequence of nucleic acid analyte(s) to that of a reference nucleic acid sequence(s).

Either the probe or the analyte, or both, may optionally incorporate more than one moiety to make up its member of the interacting label pair. The moieties may be located anywhere on the probes or analyte as long as they are capable of interacting when probe and analyte bind together. The moieties may be attached to one end of the probe or analyte, or may be attached to the interior of the probe or analyte. Members of the interacting label pairs may be attached to probes either during or post-synthesis of the probes. The attachment of a member of an interacting label pair to the probe is preferably covalent, and means of attachment will vary depending on the probe and the member of the interacting label pair, such means being readily apparent to one of skill in the art. Similar considerations apply to attachment of a probe pair to analyte.

An example of a fluorescent-quencher pair is the fluorescent moiety 5→(2-aminoethyl)aminonaphthalene-1-sulfonic acid (EDANS) and quenching moiety 4-(4-dimethylaminophenylazo)benzoic acid (DABCYL). For EDANS and DABCYL, quenching is essentially eliminated by a separation of 60 Angstroms.

All publications and patent applications cited in this specification are herein incorporated by reference as if each individual publication or patent application were specifically and individually indicated to be incorporated by reference.

Although the foregoing invention has been described in some detail by way of illustration and example for purposes of clarity of understanding, it will be readily apparent to those of ordinary skill in the art in light of the teachings of this invention that certain changes and modifications may be made thereto without departing from the spirit or scope of the appended claims.

What is claimed is:

1. A method for high throughput screening (HTS) of a compound library of one or more probes for a property of interacting with a target, the method comprising:
providing the compound library in a capillary array comprising a plurality of channels assembled in a substrate, wherein each capillary channel is capable of holding an amount of a probe and further wherein the first ends of a plurality of channels form a first face of the capillary array;
providing a reaction well adjacent one end of the capillary such that a probe in the capillary is capable of interacting with a target molecule in the reaction well;
providing at least one target molecule in the reaction well; and
detecting an interaction of a probe with the target molecule.

2. The method of claim 1, wherein the reaction well comprises a separate assay array assembled in a substrate, wherein the probe in a capillary array is capable of being in fluid communication with a channel in the assay array.

3. The method of claim 2, wherein the assay array has an identical pitch and pattern of capillaries as the capillary array.

4. The method of claim 2, wherein a first face of the assay array is coupled to the capillary array and a second face of the assay array is pneumatically coupled to a pressure chamber.

5. The method of claim 1, wherein the reaction well comprises a micro reaction well fabricated at a first end of each channel of the capillary array, wherein the probe in a capillary array is capable of being in fluid communication with the micro reaction well.

6. The method of claim 1, wherein the reaction well comprises a virtual reaction well fabricated at a first end of each channel of the capillary array, wherein the reaction well is formed on the first face of the capillary array, the reaction well being defined by a hydrophilic region at the first end of the channel and a hydrophobic region surrounding the hydrophilic region.

7. A method for screening a compound according to claim 6, wherein the reaction well has a cross-sectional area greater than a cross-sectional area of its corresponding channel.

8. The method of claim 1, wherein each capillary channel is capable of holding a metered amount of the probe.

9. The method of claim 8, wherein the probe in a solution is provided within the channel by drawing a metered amount of the probe solution into the channel by a force selected from the group consisting of a capillary force, pressure, gravity, a magnetic force and an electrical force.

10. The method of claim 1, wherein the first face of the array is accessible to liquid handling and detecting apparatus and a second distal face of the array is coupled to a pressure chamber.

11. The method of claim 1, wherein the interaction of the probe with the target is detected using an optical method.

12. The method of claim 1, wherein the substrate is a transparent material.

13. The method of claim 10, further comprising providing a target reagent to the reaction well by a fluid delivery nozzle.

14. The method of claim 10, further comprising:
pumping a probe solution to the reaction well by applying a suitable pressure differential between the pressure chamber and the first face of the array.

15. The method of claim 10, further comprising:
pumping a probe solution to the reaction well by inserting a liquid immiscible with the probe into the pressure chamber; and
moving the probe solution between the channel and the reaction well by displacing a volume of the inert fluid in the pressure chamber.

16. A method for high throughput screening (HTS) of one or more probes for an enzymatic activity, the method comprising:
(a) providing a capillary array comprising a plurality of channels assembled in a substrate, wherein each capillary channel is capable of holding an amount of a probe and further wherein the first ends of a plurality of channels form a first face of the capillary array;
(b) providing a virtual reaction well adjacent one end of the capillary, wherein the reaction well is formed on the first face of the capillary array and further wherein the reaction well is defined by a hydrophilic region at the first end of the channel and a hydrophobic region surrounding the hydrophilic region;
(c) applying a target solution to the first face of the capillary array in a flooding manner such that droplets of the target solution are retained in the reaction wells after excess solution is allowed to run off;
(d) applying a negative pressure to a pressure chamber to draw a metered amount of substrate into the channel, wherein a second distal face of the array is coupled to the pressure chamber;
(e) removing excess substrate fluid from the reaction well;
(f) applying a metered amount of an enzyme to the reaction by a method comprising steps (c) through (e) wherein the solution contains the enzyme;
(g) applying a positive pressure in the pressure chamber to push a metered amount of enzyme, target and compound into the micro-reaction well; and
(h) detecting the enzymatic activity of a probe in a channel.

17. The method of claim 16, wherein excess substrate fluid is removed from the reaction well by a method selected from the group consisting of capillary force, squeegeeing, wiping, absorption, gravity, centrifugation, air pressure, air knife blowing and vacuum force.

18. The method of claim 16, wherein the reaction is detected using optical methods.

19. The method of claim 16, wherein the substrate is transparent.

20. A desktop high throughput screening (HTS) system for detecting a property of one or more probe compounds to interact with a target, the system comprising:
(a) a compound library of probes in a capillary array comprising:
a plurality of channels assembled in a substrate, wherein each capillary channel is capable of holding an amount of a probe and further wherein the first ends of a plurality of channels form a first face of the capillary array; and
a reaction well adjacent one end of the capillary such that a probe in the capillary is capable of interacting with a target molecule in the reaction well; and
(b) a desktop HTS station comprising:
a pressure chamber capable of connecting to the capillary array;
a chamber for reacting metered amounts of probes and at least one target; and
a detector for detecting an interaction of a probe with the target molecule.

21. The system of claim 20, further comprising:
(c) a compound loading station comprising a plurality of probe compounds stored individually in a plurality of reservoirs, such that each reservoir is fluidically coupled to a channel in the capillary array.

22. The system of claim 20, wherein the reaction well comprises a separate assay array assembled in a substrate, wherein the probe in a capillary array is capable of being in fluid communication with a channel in the assay array.

23. The system of claim 20, wherein the reaction well comprises a micro reaction well fabricated at a first end of each channel of the capillary array, wherein the probe in a capillary array is capable of being in fluid communication with the micro reaction well.

24. The system of claim 20, wherein the reaction well comprises a virtual reaction well fabricated at a first end of each channel of the capillary array, wherein the reaction well is formed on the first face of the capillary array, the reaction well being defined by a hydrophilic region at the first end of the channel and a hydrophobic region surrounding the hydrophilic region.

25. The system of claim 20, wherein the desktop HTS system comprises a mechanism for removal of excess substrate fluid from the reaction well by a method selected from the group consisting of capillary force, squeegeeing, wiping, absorption, gravity, centrifugation, air pressure, air knife blowing and vacuum force.

26. The system of claim 20, wherein capillaries comprising the channels are lined with optical fiber.

27. The system of claim 20, wherein the detector detects the interaction of the target and chemical compounds by fluorescence emission, fluorescence polarization, luminescence, absorption, surface plasmon resonance (SPR).

28. The system of claim 20, wherein the detector is a CCD based imaging system, CMOS based imaging system or a scanning based fluorescence system.
